(19) United States
(12) Patent Application Publication
(10) Pub. No.: US 2024/0401042 A1
(43) Pub. Date: Dec. 5, 2024

(54) TUMOR DELIVERY OF DENDRITIC OLIGONUCLEOTIDES

(71) Applicants: UNIVERSITY OF MASSACHUSETTS, Boston, MA (US); MCGILL UNIVERSITY, Montréal (CA)

(72) Inventors: Hassan H. Fakih, Montréal (CA); Hanadi Sleiman, Montréal (CA); Anastasia Khvorova, Westborough, MA (US); John E. Harris, Sterling, MA (US); Qi Tang, Worcester, MA (US); Ken Okamura, Yamagata (JP)

(21) Appl. No.: 18/609,299

(22) Filed: Mar. 19, 2024

Related U.S. Application Data
(60) Provisional application No. 63/453,247, filed on Mar. 20, 2023.

(51) Int. Cl. C12N 15/113; A61K 47/54; A61P 35/00

(52) U.S. Cl. CPC C12N 15/113; A61K 47/548; A61K 47/549; A61P 35/00; C12N 2310/14; C12N 2310/3515

(57) ABSTRACT

This disclosure provides compositions, systems, and methods for the delivery of therapeutic oligonucleotides to a tumor. The oligonucleotide is conjugated to a dendron comprising an end group, a phosphate group, and/or a hydrophobic chain.

### CROSS REFERENCE TO RELATED APPLICATION

This application claims the benefit of U.S. Provisional Patent Application Ser. No. 63/453,247, filed Mar. 20, 2023. The entire contents of the above-referenced patent application are incorporated by reference in their entirety herein.

### STATEMENT REGARDING FEDERALLY SPONSORED RESEARCH OR DEVELOPMENT

This invention was made with government support under Grant Nos. AR069114, GM131839, and OD020012 awarded by the National Institutes of Health. The Government has certain rights in the invention.

### TECHNICAL FIELD

This disclosure relates to dendritic conjugates for the tumor delivery of therapeutic oligonucleotides. In particular, provided herein are compositions, systems, and methods for the delivery to a tumor of an oligonucleotide conjugate comprising an oligonucleotide and a dendron.

### BACKGROUND

The interaction of therapeutic oligonucleotides with proteins in body fluids has a major influence on their pharmacokinetic and pharmacodynamic properties (see Nguyen, V. H. & Lee, B.-J. “Protein corona: a new approach for nanomedicine design,” Int J Nanomedicine 12, 3137-3151 (2017); and Banker, M. J. & Clark, T. H. “Plasma/serum protein binding determinations,” Curr Drug Metab 9, 854-859 (2008), incorporated herein by reference in their entirety). Such interactions, resulting in adhesion of the proteins to oligonucleotides, alter the therapeutic's physical properties (charge, size, shape, surface chemistry) and impact their biodistribution and behavior in vivo (see Zhao, Z., Ukidve, A., Krishnan, V. & Mitragotri, S. “Effect of physicochemical and surface properties on in vivo fate of drug nanocarriers,” Advanced Drug Delivery Reviews 143, 3-21 (2019); Albanese, A., Tang, P. S. & Chan, W. C. W. “The Effect of Nanoparticle Size, Shape, and Surface Chemistry on Biological Systems,” Annual Review of Biomedical Engineering 14, 1-16 (2012); and De Castro, C. E. et al. “The Protein Corona Conundrum: Exploring the Advantages and Drawbacks of its Presence around Amphiphilic Nanoparticles,” Bioconjugate Chemistry 31, 2638-2647 (2020), incorporated herein by reference in their entirety).

Adhesion of biomolecules and proteins to the surface of therapeutics can have several advantages (see Francia, V., Schiffelers, R. M., Cullis, P. R. & Witzigmann, D. “The Biomolecular Corona of Lipid Nanoparticles for Gene Therapy,” Bioconjugate Chemistry 31, 2046-2059 (2020), incorporated herein by reference in their entirety).

The strategic design of conjugates of oligonucleotides is necessary because structure and binding affinity play a pivotal role in cellular uptake and therapeutic efficacy.

Therefore, there remains a need for novel compositions, systems, and methods for the effective delivery of therapeutic oligonucleotides.

### SUMMARY

Provided herein are compositions, systems, and methods for the delivery of an oligonucleotide conjugate comprising an oligonucleotide and a dendron. The oligonucleotide conjugates can efficient knockdown gene in tumors. Several different oligonucleotide conjugates comprising different oligonucleotides and dendron demonstrated tumor delivery upon administration.

In one aspect, the disclosure provides a method for delivering an oligonucleotide conjugate to the tumor of a subject, the method comprising administering the oligonucleotide conjugate to the subject, wherein the oligonucleotide conjugate comprises: i) an oligonucleotide comprising a 5′ and a 3′ end and complementarity to a target nucleic acid; and ii) a dendron that is linked to the oligonucleotide and comprises a end group, a phosphate group, and/or a hydrophobic chain, and wherein the oligonucleotide conjugate is formulated for tumor delivery.

In certain embodiments, the end group is a hydrophilic group comprising hydroxide, amine, phosphate, sulfur, and/or sugar; an hydrophobic group comprising amine, amide, ether, ester, N- or O-containing heterocycles, thiol, thioether, and/or saturated or unsaturated C₁₋₂₄ alkyl chain; and/or one or more aromatic rings.

In certain embodiments, the hydrophobic chain is a saturated or unsaturated C₁₋₂₄ alkyl group.

In certain embodiments, the dendron comprises two branches.

In certain embodiments, the dendron comprises four branches.

In certain embodiments, the dendron comprises eight branches.

In certain embodiments, the dendron is linked to the 5′ end and/or 3′ end of the oligonucleotide.

In certain embodiments, the oligonucleotide comprises to an antisense oligonucleotide or a siRNA.

In certain embodiments, the siRNA comprises a sense strand and an antisense strand.

In certain embodiments, the dendron is linked to the 5′ end and/or 3′ end of the sense strand or to the 5′ end and/or 3′ end of the antisense strand.

In certain embodiments, the dendron is linked to the 3′ or 5′ end of the sense strand.

In certain embodiments, the antisense strand comprises about 15 nucleotides to about 25 nucleotides in length.

In certain embodiments, the sense strand comprises about 15 nucleotides to about 25 nucleotides in length.

In certain embodiments, the antisense strand is 20 nucleotides in length, 21 nucleotides in length, or 22 nucleotides in length.

In certain embodiments, the sense strand is 15 nucleotides in length, 16 nucleotides in length, 18 nucleotides in length, or 20 nucleotides in length.

In certain embodiments, the siRNA comprises a double-stranded region of 15 base pairs to 20 base pairs.

In certain embodiments, the siRNA comprises a double-stranded region of 15 base pairs, 16 base pairs, 18 base pairs, or 20 base pairs.

In certain embodiments, the siRNA comprises at least one blunt-end.

In certain embodiments, the siRNA comprises at least one single stranded nucleotide overhang.

In certain embodiments, the siRNA comprises naturally occurring nucleotides.

In certain embodiments, the siRNA comprises at least one modified nucleotide.

In certain embodiments, the modified nucleotide comprises a 2′-O-methyl modified nucleotide, a 2′-deoxy-2′-fluoro modified nucleotide, a 2′-deoxy-modified nucleotide, a locked nucleotide, an abasic nucleotide, a 2′-amino-modified nucleotide, a 2′-alkyl-modified nucleotide, a morpholino nucleotide, a phosphoramidate, a non-natural base comprising nucleotide, or a mixture thereof.

In certain embodiments, the siRNA comprises at least one modified internucleotide linkage.

In certain embodiments, the modified internucleotide linkage comprises a phosphorothioate internucleotide linkage.

In certain embodiments, the siRNA comprises 4-16 phosphorothioate internucleotide linkages.

In certain embodiments, the siRNA comprises 8-13 phosphorothioate internucleotide linkages.

In certain embodiments, the siRNA comprises at least 80% chemically modified nucleotides.

In certain embodiments, the siRNA is fully chemically modified.

In certain embodiments, the sense strand comprises one or more nucleotide mismatches between the antisense strand and the sense strand.

In certain embodiments, the antisense strand comprises a 5′-phosphate, a 5′-alkyl phosphonate, a 5′-alkylene phosphonate, or a 5′-alkenyl phosphonate.

In certain embodiments, the antisense strand comprises a 5′-vinyl phosphonate.

In certain embodiments, the nucleotides at positions 1 and 2 from the 3′ end of sense strand, and the nucleotides at positions 1 and 2 from the 5′ end of antisense strand are connected to adjacent ribonucleotides via phosphorothioate linkages.

In certain embodiments, the oligonucleotide conjugate is administered sequentially or simultaneously.

In certain embodiments, the oligonucleotide conjugate has a structure of Formula I:

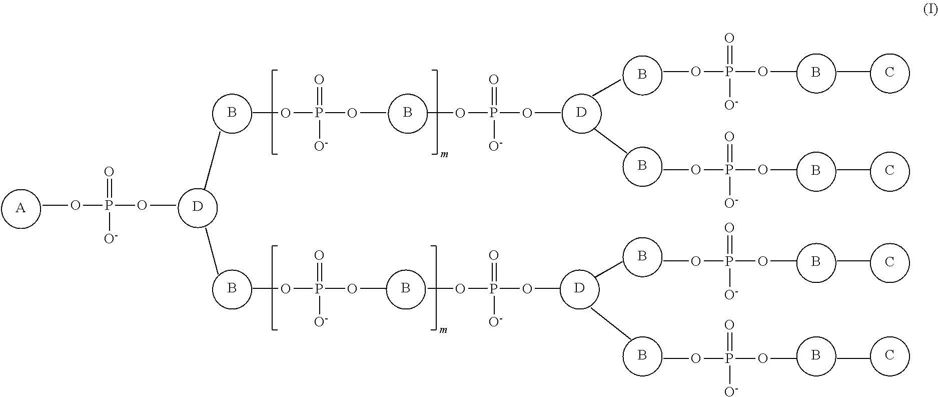

wherein:
A is an oligonucleotide;
B, for each occurrence, independently comprises one or more hydrophobic chains, amines, amides, esters, N- or O-containing heterocycles, thioethers, disulfides, and/or aromatic rings, wherein the hydrophobic chain comprises a saturated or unsaturated C₁₋₂₄ alkyl chain;
C, for each occurrence, independently comprises: an hydrophilic group comprising hydroxide, amine, phosphate, sulfur, and/or sugar; an hydrophobic group comprising amine, amide, ether, ester, N- or O-containing heterocycles, thiol, thioether, and/or saturated or unsaturated C₁₋₂₄ alkyl chain; and/or one or more aromatic rings;
D, for each occurrence, independently is a branching unit comprising one or more alkyl chains, amides, ethers, esters, and amines, wherein the branching unit comprises 2 to 4 branches; and
m, for each occurrence, independently is 0 or 1.

In certain embodiments, the oligonucleotide conjugate has a structure of Formula II:

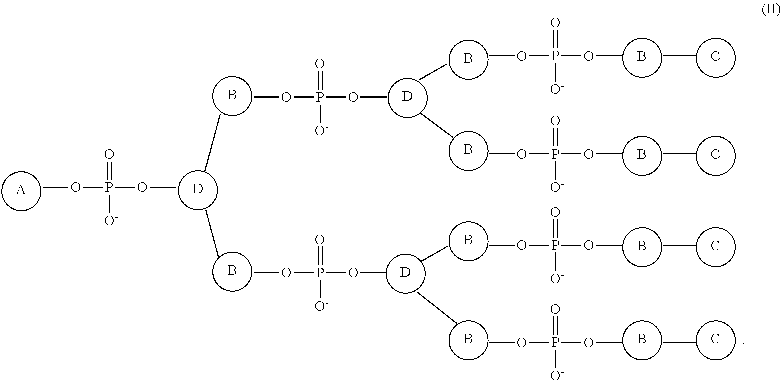

In certain embodiments, the oligonucleotide conjugate has a structure of Formula III:

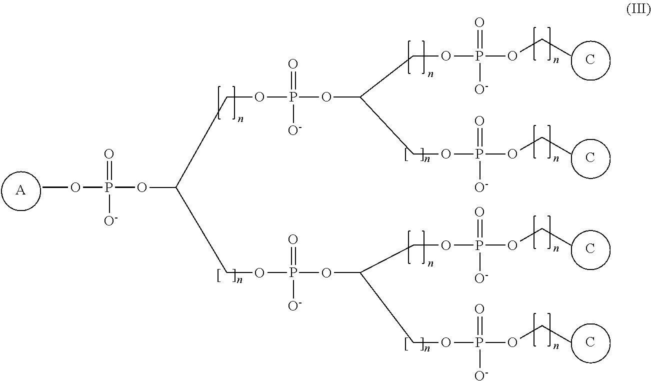

wherein n, for each occurrence, independently is an integer between 1-24.

In certain embodiments, C is OH and n, for each occurrence, independently is 1, 6, or 12.

In certain embodiments, the oligonucleotide conjugate has the structure of Formula IV:

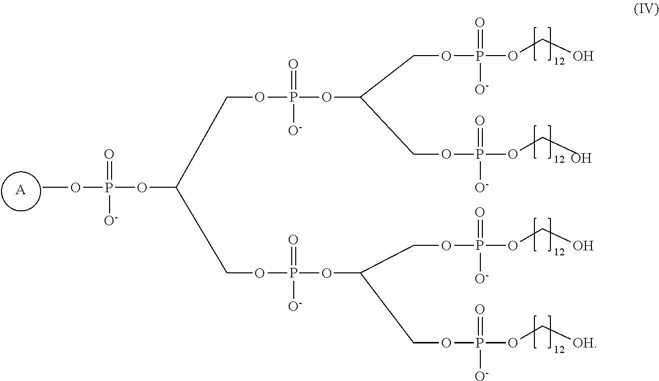

In certain embodiments, the oligonucleotide conjugate has a structure of Formula V:

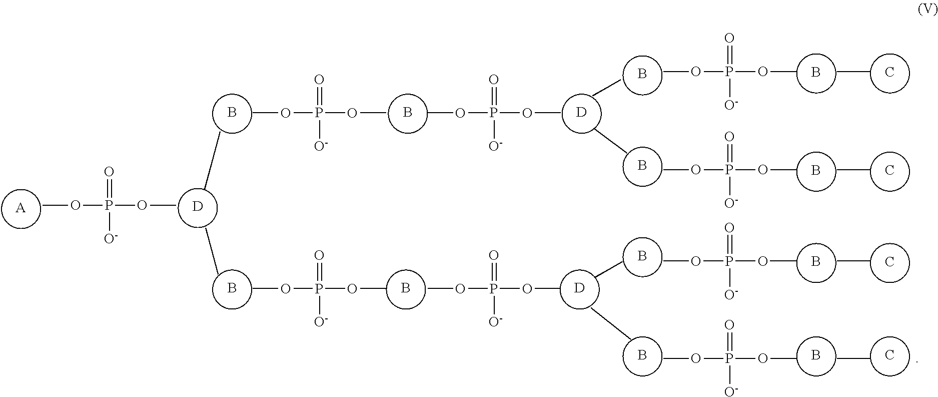

In certain embodiments, the oligonucleotide conjugate has a structure of Formula VI:

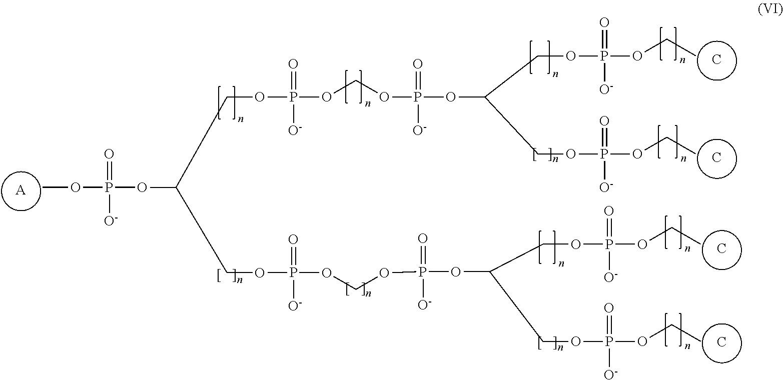

wherein n, for each occurrence, independently is an integer between 1-24.

In certain embodiments, C is OH and n, for each occurrence, independently is 1, 6, or 12.

In certain embodiments, the oligonucleotide conjugate has the structure of Formula VII:

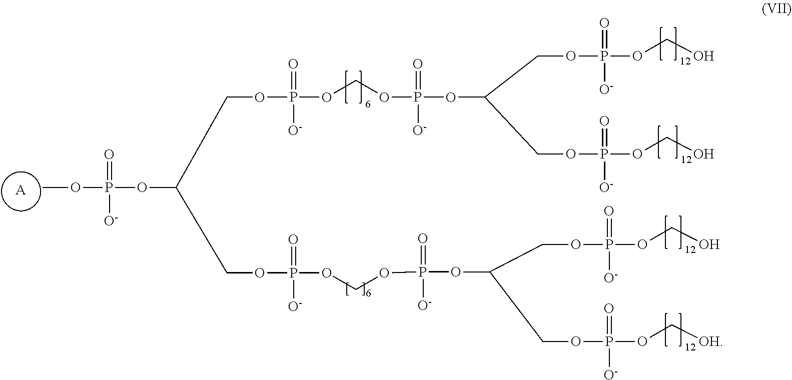

In certain embodiments, the oligonucleotide conjugate formulation comprises about 0.1-100 mg/kg of body weight of oligonucleotide conjugate.

In certain embodiments, the oligonucleotide conjugate is administered intravenous (IV) injection, subcutaneous (SQ) injection, intratumoral injection, or a combination thereof.

In certain embodiments, the oligonucleotide conjugate has low toxicity.

In certain embodiments, the oligonucleotide has a sequence of any one of the sequences of SEQ ID NOs: 10 and 11.

In one aspect, the disclosure provides a method for treating a tumor from a cancer in a patient in need of such treatment, comprising administering to the patient an oligonucleotide conjugate comprising:
i) an oligonucleotide comprising a 5′ and a 3′ end and complementarity to a target nucleic acid; and
ii) a dendron that is linked to the oligonucleotide and comprises an end group, a phosphate group, and/or a hydrophobic chain,
wherein the oligonucleotide conjugate is formulated for tumor delivery.

In some embodiments, the cancer is associated with an AIM2 gene.

In some embodiments, the oligonucleotide conjugate inhibits the expression of the AIM2 gene.

In certain embodiments, the cancer is selected from the group consisting of melanoma cancer, brain cancer, breast cancer, cervical cancer, colorectal cancer, eye cancer, fallopian tube cancer, testicular cancer, leukemia cancer, liver cancer, lung cancer, ovarian cancer, pancreatic cancer, prostate cancer, pulmonary cancer, rectal cancer, renal cancer, skin cancer, stomach cancer, and thyroid cancer.

In one aspect, the disclosure provides a method for administering a therapeutically effective amount of an oligonucleotide conjugate to the tumor a subject, the method comprising administering the oligonucleotide conjugate to the subject, wherein the oligonucleotide conjugate comprises:
i) an oligonucleotide comprising a 5′ and a 3′ end and complementarity to a target nucleic acid; and
ii) a dendron that is linked to the oligonucleotide and comprises an end group, a phosphate group, and/or a hydrophobic chain,
and wherein the oligonucleotide conjugate is formulated for tumor delivery.

In one aspect, the disclosure provides a method for inhibiting the expression of an AIM2 gene in a cell, the method comprising:
(a) introducing into the cell an oligonucleotide conjugate comprising:
i) an oligonucleotide comprising a 5′ and a 3′ end and complementarity to a target nucleic acid; and
ii) a dendron that is linked to the oligonucleotide and comprises an end group, a phosphate group, and/or a hydrophobic chain; and
(b) maintaining the cell produced in step (a) for a time sufficient to obtain degradation of the mRNA transcript of the AIM2 gene, thereby inhibiting expression of the AIM2 gene in the cell.

In one aspect, the disclosure provides an oligonucleotide conjugate for use in the treatment of a tumor in a patient in need of such treatment, wherein the oligonucleotide conjugate is administered to the patient and formulated for tumor delivery, and wherein the oligonucleotide conjugate comprises:
i) an oligonucleotide comprising a 5′ and a 3′ end and complementarity to a target nucleic acid; and
ii) a dendron that is linked to the oligonucleotide and comprises an end group, a phosphate group, and/or a hydrophobic chain.

In one aspect, the disclosure provides a pharmaceutical composition for treating a tumor in a patient in need of such treatment, comprising:
an oligonucleotide conjugate comprising:
i) an oligonucleotide comprising a 5′ and a 3′ end and complementarity to a target nucleic acid; and
ii) a dendron that is linked to the oligonucleotide and comprises an end group, a phosphate group, and/or a hydrophobic chain, and
a pharmaceutically acceptable carrier,
wherein the pharmaceutical composition is formulated for tumor delivery.

In one aspect, the disclosure provides a topical formulation comprising:
(a) an oligonucleotide conjugate comprising:
an oligonucleotide comprising a 5′ and a 3′ end and complementarity to a target nucleic acid; and
ii) a dendron that is linked to the oligonucleotide and comprises an end group, a phosphate group, and/or a hydrophobic chain;
(b) optionally a pharmaceutically acceptable carrier; and
(c) optionally a pharmaceutically acceptable salt.

In one aspect, the disclosure provides a combination comprising an oligonucleotide conjugate and an immune checkpoint inhibitor, wherein the oligonucleotide conjugate comprises:
i) an oligonucleotide comprising a 5′ and a 3′ end and complementarity to a target nucleic acid; and
ii) a dendron that is linked to the oligonucleotide and comprises an end group, a phosphate group, and/or a hydrophobic chain.

In some embodiments, the oligonucleotide comprises complementarity to an AIM2 gene or an AIM2 mRNA.

In some embodiments, the immune checkpoint inhibitor comprises an inhibitor against PD-1, PD-L1, or CTLA-4.

These and other aspects of the applicant's teachings are set forth herein.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Aspects, features, benefits, and advantages of the embodiments described herein will be apparent with regard to the following description, examples, claims, and accompanying drawings where:

FIGS. 1A-1B show schematic representations of oligonucleotide conjugates. FIG. 1A shows schematic representations of a dendron attached to a siRNA. FIG. 1B shows schematic representations of dendritic (D)-siRNAs comprising dendrons with hydrophilic (OH) and hydrophobic (CH3) end groups.

FIGS. 2A-2C show the high-performance liquid chromatography (HPLC) and size exclusion chromatography (SEC) analysis D-siRNA, DCA-siRNA, and siRNA. FIG. 2A shows the structure of the dendritic, docosanoic, and unconjugated siRNA tested for their hydrophobicity/retention time on HPLC, showing different retention times. FIG. 2B shows the reverse phase HPLC trace following the injection of D-siRNA or DCA-siRNA, showcasing the difference in hydrophobicity between them. FIG. 2C shows the results of an assay where D-siRNA, DCA-siRNA, and siRNA were injected in vivo via subcutaneous or intravenous injection in mice (n=2), and plasma was collected at 1 hour or 15 mins post injection. Plasma was run on size exclusion chromatography (SEC), Plasma proteins were monitored at 280 nm and Cy3 labeled oligonucleotides were monitored at 570 nm as previously reported.

FIG. 3A shows the administration schedule of the D-siRNA AIM2+anti-PD-1 antibody combination therapy and the tumor growth after D-siRNAAIM2 targeting injection alone or with anti-PD-1 antibody. Administration was via IV or subcutaneous route. FIG. 3B shows the administration schedule of the DCA-siRNA AIM2+anti-PD-1 antibody combination therapy and the tumor growth after DCA-siRNA AIM2 targeting injection.

FIG. 4 shows the Flow Cytometry analysis of tumor of mice injected subcutaneously with Cy3 labeled Htt Unc siRNA, DCA-siRNA, and D-siRNA. It was observed that D-siRNA increases distribution to tumor by around 2× and results in better delivery to CD45− mesenchymal cells.

FIG. 5 shows the blood chemistry and complete blood count of mice (c56bl/6, male, n=3) injected with DCA-siRNA or D-siRNA at 100 mg/Kg. Blood was collected 24 h post subcutaneous injection.

FIG. 6 shows the evaluation of efficacy in HeLa cells using RNAiMax (7-point concentration-response study) of 5′ conjugated D-siRNAs, 3′ conjugated D-siRNAs, DCA siRNA (DCA conjugate is placed on the 3′ end of the sense strand as it is grown from a solid support), and unconjugated siRNA. After 72 h, incubation samples were analyzed by Quantigene bDNA assay. Data were normalized to housekeeping gene (Hprt) and displayed as a percentage of untreated control cells. n=3. Mean±Standard deviation (SD).

### DETAILED DESCRIPTION

It will be appreciated that for clarity, the following discussion will describe various aspects of embodiments of the applicant's teachings. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s).

The disclosure relates to dendritic conjugates for the delivery of therapeutic oligonucleotides to tumor. In particular, the present disclosure provides compositions, systems, and methods for the delivery of therapeutic oligonucleotide conjugated to a dendron. The oligonucleotide conjugates disclosed herein can be delivered to the tumor upon administration.

The oligonucleotide conjugates described herein can promote simple, efficient, non-toxic delivery of oligonucleotides (e.g., siRNA, antisense oligonucleotide (ASO), macro-RNA), and promote potent silencing of therapeutic targets in tumor in vivo.

Unless otherwise specified, nomenclature used in connection with cell and tissue culture, molecular biology, immunology, microbiology, genetics and protein and nucleic acid chemistry and hybridization described herein are those well-known and commonly used in the art. Unless otherwise specified, the methods and techniques provided herein are performed according to conventional methods well known in the art and as described in various general and more specific references that are cited and discussed throughout the present specification unless otherwise indicated. Enzymatic reactions and purification techniques are performed according to manufacturer's specifications, as commonly accomplished in the art or as described herein. The nomenclature used in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, delivery, and treatment of patients.

Unless otherwise defined herein, scientific and technical terms used herein have the meanings that are commonly understood by those of ordinary skill in the art. In the event of any latent ambiguity, definitions provided herein take precedent over any dictionary or extrinsic definition. Unless otherwise required by context, singular terms shall include pluralities and plural terms shall include the singular. The use of “of” means “and/of” unless stated otherwise. The use of the term “including,” as well as other forms, such as “includes” and “included,” is not limiting.

So that the disclosure may be more readily understood, certain terms are first defined.

### Definitions

The use of the singular forms herein includes the plural unless specifically stated otherwise. As used herein, the singular forms “a,” “an,” and “the” include plural referents unless the context clearly dictates otherwise. Furthermore, use of the term “including” as well as other forms, such as “include,” “includes,” and “included,” is not limiting.

It is understood that wherever aspects are described herein with the language “comprising,” otherwise analogous aspects described in terms of “consisting of” and/or “consisting essentially of” are also provided.

As described herein, any concentration range, percentage range, ratio range or integer range is to be understood to include the value of any integer within the recited range and, when appropriate, fractions thereof (such as one tenth and one hundredth of an integer), unless otherwise indicated.

The terms “about” or “comprising essentially of” refer to a value or composition that is within an acceptable error range for the particular value or composition as determined by one of ordinary skill in the art, which will depend in part on how the value or composition is measured or determined, i.e., the limitations of the measurement system. When particular values or compositions are provided in the application and claims, unless otherwise stated, the meaning of “about” or “comprising essentially of” should be assumed to be within an acceptable error range for that particular value or composition.

The term “and/of” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

As used herein in the context of oligonucleotide sequences, “A” represents a nucleoside comprising the base adenine (e.g., adenosine or a chemically-modified derivative thereof), “G” represents a nucleoside comprising the base guanine (e.g., guanosine or a chemically-modified derivative thereof), “U” represents a nucleoside comprising the base uracil (e.g., uridine or a chemically-modified derivative thereof), and “C” represents a nucleoside comprising the base adenine (e.g., cytidine or a chemically-modified derivative thereof).

The term “nucleoside” refers to a molecule having a purine or pyrimidine base covalently linked to a ribose or deoxyribose sugar. Exemplary nucleosides include adenosine, guanosine, cytidine, uridine and thymidine. Additional exemplary nucleosides include inosine, 1-methyl inosine, pseudouridine, 5,6-dihydrouridine, ribothymidine, 2N-methylguanosine and N2,N2-dimethylguanosine (also referred to as “rare” nucleosides). The term “nucleotide” refers to a nucleoside having one or more phosphate groups joined in ester linkages to the sugar moiety. Exemplary nucleotides include nucleoside monophosphates, diphosphates and triphosphates. The terms “polynucleotide” and “nucleic acid molecule” are used interchangeably herein and refer to a polymer of nucleotides joined together by a phosphodiester or phosphorothioate linkage between 5′ and 3′ carbon atoms.

The term “RNA” or “RNA molecule” or “ribonucleic acid molecule” refers to a polymer of ribonucleotides (e.g., 2, 3, 4, 5, 10, 15, 20, 25, 30, or more ribonucleotides). The term “DNA” or “DNA molecule” or “deoxyribonucleic acid molecule” refers to a polymer of deoxyribonucleotides. DNA and RNA can be synthesized naturally (e.g., by DNA replication or transcription of DNA, respectively). RNA can be post-transcriptionally modified. DNA and RNA can also be chemically synthesized. DNA and RNA can be single-stranded (i.e., ssRNA and ssDNA, respectively) or multi-stranded (e.g., double stranded, i.e., dsRNA and dsDNA, respectively). “mRNA” or “messenger RNA” is single-stranded RNA that specifies the amino acid sequence of one or more polypeptide chains. This information is translated during protein synthesis when ribosomes bind to the mRNA.

As used herein, the term “small interfering RNA” (“siRNA”) (also referred to in the art as “short interfering RNAs”) refers to an RNA (or RNA analog) comprising between about 10-50 nucleotides (or nucleotide analogs), which is capable of directing or mediating RNA interference. The siRNA is a duplex formed by a sense strand and antisense strand which have sufficient complementarity to each other to form said duplex. In certain embodiments, a siRNA comprises between about 15-30 nucleotides or nucleotide analogs, or between about 16-25 nucleotides (or nucleotide analogs), or between about 18-23 nucleotides (or nucleotide analogs), or between about 19-22 nucleotides (or nucleotide analogs) (e.g., 19, 20, 21 or 22 nucleotides or nucleotide analogs). The term “short” siRNA refers to a siRNA comprising about 21 nucleotides (or nucleotide analogs), for example, 19, 20, 21 or 22 nucleotides. The term “long” siRNA refers to a siRNA comprising about 24-25 nucleotides, for example, 23, 24, 25 or 26 nucleotides. Short siRNAs may, in some instances, include fewer than 19 nucleotides, e.g., 16, 17 or 18 nucleotides, provided that the shorter siRNA retains the ability to mediate RNAi. Likewise, long siRNAs may, in some instances, include more than 26 nucleotides, provided that the longer siRNA retains the ability to mediate RNAi absent further processing, e.g., enzymatic processing, to a short siRNA.

The term “nucleotide analog” or “altered nucleotide” or “modified nucleotide” or “chemically modified nucleotide” refers to a non-standard nucleotide, including non-naturally occurring ribonucleotides or deoxyribonucleotides. Exemplary nucleotide analogs are modified at any position so as to alter certain chemical properties of the nucleotide yet retain the ability of the nucleotide analog to perform its intended function. Examples of positions of the nucleotide, which may be derivatized include: the 5 position, e.g., 5-(2-amino)propyl uridine, 5-bromo uridine, 5-propyne uridine, 5-propenyl uridine, etc.; the 6 position, e.g., 6-(2-amino)propyl uridine; and the 8-position for adenosine and/or guanosines, e.g., 8-bromo guanosine, 8-chloro guanosine, 8-fluoroguanosine, etc. Nucleotide analogs also include deaza nucleotides, e.g., 7-deaza-adenosine; O- and N-modified (e.g., alkylated, e.g., N6-methyl adenosine, or as otherwise known in the art) nucleotides; and other heterocyclically modified nucleotide analogs, such as those described in Herdewijn, Antisense Nucleic Acid Drug Dev., 2000 Aug. 10(4):297-310.

Nucleotide analogs may also comprise modifications to the sugar portion of the nucleotides. For example, the 2′ OH-group may be replaced by a group selected from H, OR, R, F, Cl, Br, I, SH, SR, NH₂, NHR, NR₂, or COOR, wherein R is substituted or unsubstituted C₁-C₆ alkyl, alkenyl, alkynyl, aryl, etc. Other possible modifications include those described in U.S. Pat. Nos. 5,858,988, and 6,291,438. In certain embodiments, the nucleotide analog comprises a 2′-O-methyl modification. In certain embodiments, the nucleotide analog comprises a 2′-fluoro modification.

The phosphate group of the nucleotide may also be modified, e.g., by substituting one or more of the oxygens of the phosphate group with sulfur (e.g., phosphorothioate), or by making other substitutions, which allow the nucleotide to perform its intended function, such as described in, for example, Eckstein, Antisense Nucleic Acid Drug Dev. 2000 Apr. 10(2):117-21, Rusckowski et al. Antisense Nucleic Acid Drug Dev. 2000 Oct. 10(5):333-45, Stein, Antisense Nucleic Acid Drug Dev. 2001 Oct. 11(5): 317-25, Vorobjev et al. Antisense Nucleic Acid Drug Dev. 2001 Apr. 11(2):77-85, and U.S. Pat. No. 5,684,143. Certain of the above-referenced modifications (e.g., phosphate group modifications) decrease the rate of hydrolysis of, for example, polynucleotides comprising said analogs in vivo or in vitro.

The term “RNA analog” refers to a polynucleotide (e.g., a chemically synthesized polynucleotide) having at least one altered or modified nucleotide as compared to a corresponding unaltered or unmodified RNA, but retaining the same or similar nature or function as the corresponding unaltered or unmodified RNA. As discussed above, the oligonucleotides may be linked with linkages, which result in a lower rate of hydrolysis of the RNA analog as compared to an RNA molecule with phosphodiester linkages. For example, the nucleotides of the analog may comprise methylenediol, ethylene diol, oxymethylthio, oxyethylthio, oxycarbonyloxy, phosphorodiamidate, phosphoroamidate, and/or phosphorothioate linkages. Some RNA analogues include sugar- and/or backbone-modified ribonucleotides and/or deoxyribonucleotides. Such alterations or modifications can further include addition of non-nucleotide material, such as to the end(s) of the RNA or internally (at one or more nucleotides of the RNA). An RNA analog need only be sufficiently similar to natural RNA that it has the ability to mediate RNA interference.

As used herein, the term “RNA interference” (“RNAi”) refers to a selective intracellular degradation of RNA. RNAi occurs in cells naturally to remove foreign RNAs (e.g., viral RNAs). Natural RNAi proceeds via fragments cleaved from free dsRNA, which direct the degradative mechanism to other similar RNA sequences. Alternatively, RNAi can be initiated by the hand of man, for example, to silence the expression of target genes.

An RNAi agent, e.g., an RNA silencing agent, having a strand, which is “sequence sufficiently complementary to a target mRNA sequence to direct target-specific RNA interference (RNAi)” means that the strand has a sequence sufficient to trigger the destruction of the target mRNA by the RNAi machinery or process.

As used herein, the term “isolated RNA” (e.g., “isolated siRNA” or “isolated siRNA precursor”) refers to RNA molecules, which are substantially free of other cellular material, or culture medium when produced by recombinant techniques, or substantially free of chemical precursors or other chemicals when chemically synthesized.

As used herein, the term “RNA silencing” refers to a group of sequence-specific regulatory mechanisms (e.g., RNA interference (RNAi), transcriptional gene silencing (TGS), post-transcriptional gene silencing (PTGS), quelling, co-suppression, and translational repression) mediated by RNA molecules, which result in the inhibition or “silencing” of the expression of a corresponding protein-coding gene. RNA silencing has been observed in many types of organisms, including plants, animals, and fungi.

The term “in vitro” has its art recognized meaning, e.g., involving purified reagents or extracts, e.g., cell extracts. The term “in vivo” also has its art recognized meaning, e.g., involving living cells, e.g., immortalized cells, primary cells, cell lines, and/or cells in an organism.

As used herein, a “target” refers to a particular nucleic acid sequence (e.g., a gene, an mRNA, a miRNA or the like) that an oligonucleotide conjugate or branched oligonucleotide of the disclosure binds to and/or otherwise effects the expression of. In certain embodiments, the target is expressed in the eye. In certain embodiments, target is expressed in a specific eye cell. In other embodiments, a target is associated with a particular disease or disorder in a subject.

As used herein, the term “target gene” is a gene whose expression is to be substantially inhibited or “silenced.” This silencing can be achieved by RNA silencing, e.g., by cleaving the mRNA of the target gene or translational repression of the target gene. The term “non-target gene” is a gene whose expression is not to be substantially silenced. In one embodiment, the polynucleotide sequences of the target and non-target gene (e.g., mRNA encoded by the target and non-target genes) can differ by one or more nucleotides. In another embodiment, the target and non-target genes can differ by one or more polymorphisms (e.g., Single Nucleotide Polymorphisms or SNPs). In another embodiment, the target and non-target genes can share less than 100% sequence identity. In another embodiment, the non-target gene may be a homologue (e.g., an orthologue or paralogue) of the target gene.

As used herein, the term “RNA silencing agent” refers to an RNA, which is capable of inhibiting or “silencing” the expression of a target gene. In certain embodiments, the RNA silencing agent is capable of preventing complete processing (e.g., the full translation and/or expression) of a mRNA molecule through a post-transcriptional silencing mechanism. RNA silencing agents include small (<50 b.p.), noncoding RNA molecules, for example RNA duplexes comprising paired strands, as well as precursor RNAs from which such small non-coding RNAs can be generated. Exemplary RNA silencing agents include siRNAs, miRNAs, siRNA-like duplexes, antisense oligonucleotides, GAPMER molecules, and dual-function oligonucleotides, as well as precursors thereof. In one embodiment, the RNA silencing agent is capable of inducing RNA interference. In another embodiment, the RNA silencing agent is capable of mediating translational repression.

As used herein, the term “rare nucleotide” refers to a naturally occurring nucleotide that occurs infrequently, including naturally occurring deoxyribonucleotides or ribonucleotides that occur infrequently, e.g., a naturally occurring ribonucleotide that is not guanosine, adenosine, cytosine, or uridine. Examples of rare nucleotides include, but are not limited to, inosine, 1-methyl inosine, pseudouridine, 5,6-dihydrouridine, ribothymidine, 2N-methylguanosine and 2,2N,N-dimethylguanosine.

The term “engineered,” as in an engineered RNA precursor, or an engineered nucleic acid molecule, indicates that the precursor or molecule is not found in nature, in that all or a portion of the nucleic acid sequence of the precursor or molecule is created or selected by a human. Once created or selected, the sequence can be replicated, translated, transcribed, or otherwise processed by mechanisms within a cell. Thus, an RNA precursor produced within a cell from a transgene that includes an engineered nucleic acid molecule is an engineered RNA precursor.

As used herein, the term “microRNA” (“miRNA”), also known in the art as “small temporal RNAs” (“stRNAs”), refers to a small (10-50 nucleotide) RNA, which are genetically encoded (e.g., by viral, mammalian, or plant genomes) and are capable of directing or mediating RNA silencing. An “miRNA disorder” shall refer to a disease or disorder characterized by an aberrant expression or activity of a miRNA.

As used herein, the term “dual functional oligonucleotide” refers to an RNA silencing agent having the formula T-L-μ, wherein T is an mRNA targeting moiety, L is a linking moiety, and p is a miRNA recruiting moiety. As used herein, the terms “mRNA targeting moiety,” “targeting moiety,” “mRNA targeting portion” or “targeting portion” refer to a domain, portion or region of the dual functional oligonucleotide having sufficient size and sufficient complementarity to a portion or region of an mRNA chosen or targeted for silencing (i.e., the moiety has a sequence sufficient to capture the target mRNA).

As used herein, the term “linking moiety” or “linking portion” refers to a domain, portion or region of the RNA-silencing agent which covalently joins or links the mRNA.

As used herein, the term “antisense strand” of an RNA silencing agent, e.g., an siRNA, refers to a strand that is substantially complementary to a section of about 10-50 nucleotides, e.g., about 15-30, 16-25, 18-23 or 19-22 nucleotides of the mRNA of the gene targeted for silencing. The antisense strand or first strand has sequence sufficiently complementary to the desired target mRNA sequence to direct target-specific silencing, e.g., complementarity sufficient to trigger the destruction of the desired target mRNA by the RNAi machinery or process (RNAi interference) or complementarity sufficient to trigger translational repression of the desired target mRNA.

The term “sense strand” or “second strand” of an RNA silencing agent, e.g., an siRNA or RNA silencing agent, refers to a strand that is complementary to the antisense strand or first strand. Antisense and sense strands can also be referred to as first or second strands, the first or second strand having complementarity to the target sequence and the respective second or first strand having complementarity to said first or second strand. miRNA duplex intermediates or siRNA-like duplexes include a miRNA strand having sufficient complementarity to a section of about 10-50 nucleotides of the mRNA of the gene targeted for silencing and a miRNA* strand having sufficient complementarity to form a duplex with the miRNA strand.

As used herein, the term “guide strand” refers to a strand of an RNA silencing agent, e.g., an antisense strand of an siRNA duplex or siRNA sequence, that enters into the RISC complex and directs cleavage of the target mRNA.

As used herein, the term “asymmetry,” as in the asymmetry of the duplex region of an RNA silencing agent (e.g., the stem of an shRNA), refers to an inequality of bond strength or base pairing strength between the termini of the RNA silencing agent (e.g., between terminal nucleotides on a first strand or stem portion and terminal nucleotides on an opposing second strand or stem portion), such that the 5′ end of one strand of the duplex is more frequently in a transient unpaired, e.g., single-stranded, state than the 5′ end of the complementary strand. This structural difference determines that one strand of the duplex is preferentially incorporated into a RISC complex. The strand whose 5′ end is less tightly paired to the complementary strand will preferentially be incorporated into RISC and mediate RNAi.

As used herein, the term “bond strength” or “base pair strength” refers to the strength of the interaction between pairs of nucleotides (or nucleotide analogs) on opposing strands of an oligonucleotide duplex (e.g., an siRNA duplex), due primarily to H-bonding, van der Waals interactions, and the like, between said nucleotides (or nucleotide analogs).

As used herein, the “5′ end,” as in the 5′ end of an antisense strand, refers to the 5′ terminal nucleotides, e.g., between one and about 5 nucleotides at the 5′ terminus of the antisense strand. As used herein, the “3′ end,” as in the 3′ end of a sense strand, refers to the region, e.g., a region of between one and about 5 nucleotides, that is complementary to the nucleotides of the 5′ end of the complementary antisense strand.

As used herein the term “destabilizing nucleotide” refers to a first nucleotide or nucleotide analog capable of forming a base pair with second nucleotide or nucleotide analog such that the base pair is of lower bond strength than a conventional base pair (i.e., Watson-Crick base pair). In certain embodiments, the destabilizing nucleotide is capable of forming a mismatch base pair with the second nucleotide. In other embodiments, the destabilizing nucleotide is capable of forming a wobble base pair with the second nucleotide. In yet other embodiments, the destabilizing nucleotide is capable of forming an ambiguous base pair with the second nucleotide.

As used herein, the term “base pair” refers to the interaction between pairs of nucleotides (or nucleotide analogs) on opposing strands of an oligonucleotide duplex (e.g., a duplex formed by a strand of an RNA silencing agent and a target mRNA sequence), due primarily to H-bonding, van der Waals interactions, and the like between said nucleotides (or nucleotide analogs). As used herein, the term “bond strength” or “base pair strength” refers to the strength of the base pair.

As used herein, the term “mismatched base pair” refers to a base pair consisting of non-complementary or non-Watson-Crick base pairs, for example, not normal complementary G:C, A:T or A:U base pairs. As used herein the term “ambiguous base pair” (also known as a non-discriminatory base pair) refers to a base pair formed by a universal nucleotide.

As used herein, term “universal nucleotide” (also known as a “neutral nucleotide”) include those nucleotides (e.g., certain destabilizing nucleotides) having a base (a “universal base” or “neutral base”) that does not significantly discriminate between bases on a complementary polynucleotide when forming a base pair. Universal nucleotides are predominantly hydrophobic molecules that can pack efficiently into antiparallel duplex nucleic acids (e.g., double-stranded DNA or RNA) due to stacking interactions. The base portion of universal nucleotides typically comprise a nitrogen-containing aromatic heterocyclic moiety.

As used herein, the terms “sufficient complementarity” or “sufficient degree of complementarity” mean that the RNA silencing agent has a sequence (e.g., in the antisense strand, mRNA targeting moiety or miRNA recruiting moiety), which is sufficient to bind the desired target RNA, respectively, and to trigger the RNA silencing of the target mRNA.

As used herein, the term “translational repression” refers to a selective inhibition of mRNA translation. Natural translational repression proceeds via miRNAs cleaved from shRNA precursors. Both RNAi and translational repression are mediated by RISC. Both RNAi and translational repression occur naturally or can be initiated by the hand of man, for example, to silence the expression of target genes.

As used herein, the term “toxicity” refers to a diverse array of adverse effects which are brought about through the use of the oligonucleotide conjugate of the disclosure, at either therapeutic or non-therapeutic doses. Toxicity may be measured in a subject who has been administered the oligonucleotide conjugate by measuring one or more biological parameters of the subject. For example, but in no way limiting, a subject may have blood chemistry and complete blood counts (CBC) determined before and after administration of the oligonucleotide conjugate. Changes in blood chemistry of CBC may be indicative of toxicity. Blood chemistry includes, but is not limited to, measurement of liver enzymes (e.g., Alanine transaminase (ALT), Aspartate transaminase (AST), Alkaline phosphatase (ALP)), measurement of albumin, and/or measurement of amylase. An additional measurement of toxicity may be the uptake of the oligonucleotide conjugate of the disclosure into immune cells, with higher uptake being considered a sign of increased toxicity. Such immune cell uptake may lead to increased inflammatory cytokine production.

Various methodologies of the instant disclosure include a step that involves comparing a value, level, feature, characteristic, property, etc. to a “suitable control,” referred to interchangeably herein as an “appropriate control.” A “suitable control” or “appropriate control” is any control or standard familiar to one of ordinary skill in the art useful for comparison purposes. In one embodiment, a “suitable control” or “appropriate control” is a value, level, feature, characteristic, property, etc. determined prior to performing an RNAi methodology, as described herein. For example, a transcription rate, mRNA level, translation rate, protein level, biological activity, cellular characteristic or property, genotype, phenotype, etc. can be determined prior to introducing an RNA silencing agent of the disclosure into a cell or organism. In another embodiment, a “suitable control” or “appropriate control” is a value, level, feature, characteristic, property, etc. determined in a cell or organism, e.g., a control or normal cell or organism, exhibiting, for example, normal traits. In yet another embodiment, a “suitable control” or “appropriate control” is a predefined value, level, feature, characteristic, property, etc.

Design of siRNA Molecules

The oligonucleotide conjugates described herein comprise an oligonucleotide. Non-limiting examples of oligonucleotide include siRNA, antisense oligonucleotide (ASO), and macro-RNA.

In some embodiments, an siRNA molecule of the invention is a duplex consisting of a sense strand and complementary antisense strand, the antisense strand having sufficient complementary to a target sequence such as a mRNA sequence (e.g., a htt mRNA sequence, cyclophilin B mRNA sequence, etc.) to mediate RNAi. Preferably, the siRNA molecule has a length from about 10-50 or more nucleotides, i.e., each strand comprises 10-50 nucleotides (or nucleotide analogs). More preferably, the siRNA molecule has a length from about 16-30, e.g., 16, 17, 18, 19, 20, 21, 22, 23, 24, 25, 26, 27, 28, 29, or 30 nucleotides in each strand, wherein one of the strands is sufficiently complementary to a target region. Preferably, the strands are aligned such that there are at least 1, 2, or 3 bases at the end of the strands which do not align (i.e., for which no complementary bases occur in the opposing strand) such that an overhang of 1, 2 or 3 residues occurs at one or both ends of the duplex when strands are annealed. Preferably, the siRNA molecule has a length from about 10-50 or more nucleotides, i.e., each strand comprises 10-50 nucleotides (or nucleotide analogs). More preferably, the siRNA molecule has a length from about 16-30, e.g., 16, 17, 18, 19, 20, 21, 22, 23, 24, 25, 26, 27, 28, 29, or 30 nucleotides in each strand, wherein one of the strands is substantially complementary to a target sequence, and the other strand is identical or substantially identical to the first strand.

Generally, siRNAs can be designed by using any method known in the art, for instance, by using the following protocol:

1. The siRNA may be specific for a target sequence. Preferably, the first strand is substantially complementary to the target sequence, and the other strand is substantially complementary to the first strand. In an embodiment, the target sequence is outside a coding region of the target gene. Exemplary target sequences are selected from the 5′ untranslated region (5′-UTR) or an intronic region of a target gene. Cleavage of mRNA at these sites should eliminate translation of corresponding mutant protein. Target sequences from other regions of the htt gene are also suitable for targeting. A sense strand is designed based on the target sequence. Further, siRNAs with lower G/C content (35-55%) may be more active than those with G/C content higher than 55%. Thus, in one embodiment, the invention includes nucleic acid molecules having 35-55% G/C content.

2. The sense strand of the siRNA is designed based on the sequence of the selected target site. Preferably the RNA silencing agents of the invention do not elicit a PKR response (i.e., are of a sufficiently short length). However, longer RNA silencing agents may be useful, for example, in cell types incapable of generating a PRK response or in situations where the PKR response has been down-regulated or dampened by alternative means.

The siRNA molecules of the invention have sufficient complementarity with the target sequence such that the siRNA can mediate RNAi. In general, siRNA containing nucleotide sequences sufficiently identical to a target sequence portion of the target gene to effect RISC-mediated cleavage of the target gene are preferred. Accordingly, in a preferred embodiment, the sense strand of the siRNA is designed have to have a sequence sufficiently identical to a portion of the target. For example, the sense strand may have 100% identity to the target site. However, 100% identity is not required. Greater than 80% identity, e.g., 80%, 81%, 82%, 83%, 84%, 85%, 86%, 87%, 88%, 89%, 90%, 91%, 92%, 93%, 94%, 95%, 96%, 97%, 98%, 99% or even 100% identity, between the sense strand and the target RNA sequence is preferred. The invention has the advantage of being able to tolerate certain sequence variations to enhance efficiency and specificity of RNAi. In one embodiment, the sense strand has 4, 3, 2, 1, or 0 mismatched nucleotide(s) with a target region, such as a target region that differs by at least one base pair between a wild-type and mutant allele, e.g., a target region comprising the gain-of-function mutation, and the other strand is identical or substantially identical to the first strand. Moreover, siRNA sequences with small insertions or deletions of 1 or 2 nucleotides may also be effective for mediating RNAi. Alternatively, siRNA sequences with nucleotide analog substitutions or insertions can be effective for inhibition.

Sequence identity may be determined by sequence comparison and alignment algorithms known in the art. To determine the percent identity of two nucleic acid sequences (or of two amino acid sequences), the sequences are aligned for optimal comparison purposes (e.g., gaps can be introduced in the first sequence or second sequence for optimal alignment). The nucleotides (or amino acid residues) at corresponding nucleotide (or amino acid) positions are then compared. When a position in the first sequence is occupied by the same residue as the corresponding position in the second sequence, then the molecules are identical at that position. The percent identity between the two sequences is a function of the number of identical positions shared by the sequences (i.e., percent (%) homology=number of identical positions/total number of positions×100), optionally penalizing the score for the number of gaps introduced and/or length of gaps introduced.

The comparison of sequences and determination of percent identity between two sequences can be accomplished using a mathematical algorithm. In one embodiment, the alignment generated over a certain portion of the sequence aligned having sufficient identity but not over portions having low degree of identity (i.e., a local alignment). A preferred, non-limiting example of a local alignment algorithm utilized for the comparison of sequences is the algorithm of Karlin and Altschul (1990) Proc. Natl. Acad. Sci. USA 87:2264-68, modified as in Karlin and Altschul (1993) Proc. Natl. Acad. Sci. USA 90:5873-77. Such an algorithm is incorporated into the BLAST programs (version 2.0) of Altschul, et al. (1990) J. Mol. Biol. 215:403-10.

In another embodiment, the alignment is optimized by introducing appropriate gaps and percent identity is determined over the length of the aligned sequences (i.e., a gapped alignment). To obtain gapped alignments for comparison purposes, Gapped BLAST can be utilized as described in Altschul et al., (1997) Nucleic Acids Res. 25(17):3389-3402. In another embodiment, the alignment is optimized by introducing appropriate gaps and percent identity is determined over the entire length of the sequences aligned (i.e., a global alignment). A preferred, non-limiting example of a mathematical algorithm utilized for the global comparison of sequences is the algorithm of Myers and Miller, CABIOS (1989). Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

3. The antisense or guide strand of the siRNA is routinely the same length as the sense strand and includes complementary nucleotides. In one embodiment, the guide and sense strands are fully complementary, i.e., the strands are blunt ended when aligned or annealed. In another embodiment, the strands of the siRNA can be paired in such a way as to have a 3′ overhang of 1 to 4, e.g., 2, nucleotides. Overhangs can comprise (or consist of) nucleotides corresponding to the target gene sequence (or complement thereof). Alternatively, overhangs can comprise (or consist of) deoxyribonucleotides, for example dTs, or nucleotide analogs, or other suitable non-nucleotide material. Thus, in another embodiment, the nucleic acid molecules may have a 3′ overhang of 2 nucleotides, such as TT. The overhanging nucleotides may be either RNA or DNA. As noted above, it is desirable to choose a target region wherein the mutant:wild type mismatch is a purine:purine mismatch.

4. Using any method known in the art, compare the potential targets to the appropriate genome database (human, mouse, rat, etc.) and eliminate from consideration any target sequences with significant homology to other coding sequences. One such method for such sequence homology searches is known as BLAST, which is available at National Center for Biotechnology Information website.

5. Select one or more sequences that meet the criteria for evaluation.

Further general information about the design and use of siRNA may be found in “The siRNA User Guide,” available at The Max-Plank-Institut fur Biophysikalishe Chemie website.

Alternatively, the siRNA may be defined functionally as a nucleotide sequence (or oligonucleotide sequence) that is capable of hybridizing with the target sequence (e.g., 400 mM NaCl, 40 mM PIPES pH 6.4, 1 mM EDTA, 50° C. or 70° C. hybridization for 12-16 hours; followed by washing). Additional preferred hybridization conditions include hybridization at 70° C. in 1×SSC or 50° C. in 1×SSC, 50% formamide followed by washing at 70° C. in 0.3×SSC or hybridization at 70° C. in 4×SSC or 50° C. in 4×SSC, 50% formamide followed by washing at 67° C. in 1×SSC. The hybridization temperature for hybrids anticipated to be less than 50 base pairs in length should be 5-10° C. less than the melting temperature (Tm) of the hybrid, where Tm is determined according to the following equations. For hybrids less than 18 base pairs in length, Tm(° C.)=2(# of A+T bases)+4(# of G+C bases). For hybrids between 18 and 49 base pairs in length, Tm(° C.)=81.5+16.6(log 10[Na+])+0.41(% G+C)−(600/N), where N is the number of bases in the hybrid, and [Na+] is the concentration of sodium ions in the hybridization buffer ([Na+] for 1×SSC=0.165 M). Additional examples of stringency conditions for polynucleotide hybridization are provided in Sambrook, J., E. F. Fritsch, and T. Maniatis, 1989, Molecular Cloning: A Laboratory Manual, Cold Spring Harbor Laboratory Press, Cold Spring Harbor, N.Y., chapters 9 and 11, and Current Protocols in Molecular Biology, 1995, F. M. Ausubel et al., eds., John Wiley & Sons, Inc., sections 2.10 and 6.3-6.4, incorporated herein by reference.

Negative control siRNAs should have the same nucleotide composition as the selected siRNA, but without significant sequence complementarity to the appropriate genome. Such negative controls may be designed by randomly scrambling the nucleotide sequence of the selected siRNA. A homology search can be performed to ensure that the negative control lacks homology to any other gene in the appropriate genome. In addition, negative control siRNAs can be designed by introducing one or more base mismatches into the sequence.

6. To validate the effectiveness by which siRNAs destroy target mRNAs (e.g., wild-type or mutant huntingtin mRNA), the siRNA may be incubated with target cDNA (e.g., huntingtin cDNA) in a Drosophila-based in vitro mRNA expression system. Radiolabeled with ³²P, newly synthesized target mRNAs (e.g., huntingtin mRNA) are detected autoradiographically on an agarose gel. The presence of cleaved target mRNA indicates mRNA nuclease activity. Suitable controls include omission of siRNA and use of non-target cDNA. Alternatively, control siRNAs are selected having the same nucleotide composition as the selected siRNA, but without significant sequence complementarity to the appropriate target gene. Such negative controls can be designed by randomly scrambling the nucleotide sequence of the selected siRNA. A homology search can be performed to ensure that the negative control lacks homology to any other gene in the appropriate genome. In addition, negative control siRNAs can be designed by introducing one or more base mismatches into the sequence.

siRNAs may be designed to target any of the target sequences described supra. Said siRNAs comprise an antisense strand which is sufficiently complementary with the target sequence to mediate silencing of the target sequence. In certain embodiments, the RNA silencing agent is a siRNA.

Sites of siRNA-mRNA complementation are selected which result in optimal mRNA specificity and maximal mRNA cleavage.

siRNA-Like Molecules

siRNA-like molecules of the invention have a sequence (i.e., have a strand having a sequence) that is “sufficiently complementary” to a target sequence of an mRNA (e.g., htt mRNA) to direct gene silencing either by RNAi or translational repression. siRNA-like molecules are designed in the same way as siRNA molecules, but the degree of sequence identity between the sense strand and target RNA approximates that observed between an miRNA and its target. In general, as the degree of sequence identity between a miRNA sequence and the corresponding target gene sequence is decreased, the tendency to mediate post-transcriptional gene silencing by translational repression rather than RNAi is increased. Therefore, in an alternative embodiment, where post-transcriptional gene silencing by translational repression of the target gene is desired, the miRNA sequence has partial complementarity with the target gene sequence. In certain embodiments, the miRNA sequence has partial complementarity with one or more short sequences (complementarity sites) dispersed within the target mRNA (e.g., within the 3′-UTR of the target mRNA) (Hutvagner and Zamore, Science, 2002; Zeng et al., Mol. Cell, 2002; Zeng et al., RNA, 2003; Doench et al., Genes & Dev., 2003). Since the mechanism of translational repression is cooperative, multiple complementarity sites (e.g., 2, 3, 4, 5, or 6) may be targeted in certain embodiments.

The capacity of a siRNA-like duplex to mediate RNAi or translational repression may be predicted by the distribution of non-identical nucleotides between the target gene sequence and the nucleotide sequence of the silencing agent at the site of complementarity. In one embodiment, where gene silencing by translational repression is desired, at least one non-identical nucleotide is present in the central portion of the complementarity site so that duplex formed by the miRNA guide strand and the target mRNA contains a central “bulge” (Doench J G et al., Genes & Dev., 2003). In another embodiment 2, 3, 4, 5, or 6 contiguous or non-contiguous non-identical nucleotides are introduced. The non-identical nucleotide may be selected such that it forms a wobble base pair (e.g., G:U) or a mismatched base pair (G:A, C:A, C:U, G:G, A:A, C:C, U:U). In a further preferred embodiment, the “bulge” is centered at nucleotide positions 12 and 13 from the 5′ end of the miRNA molecule.

### Modified RNA Silencing Agents

In certain aspects of the invention, an RNA silencing agent (or any portion thereof) of the invention as described supra may be modified such that the activity of the agent is further improved. For example, the RNA silencing agents described in above may be modified with any of the modifications described infra. The modifications can, in part, serve to further enhance target discrimination, to enhance stability of the agent (e.g., to prevent degradation), to promote cellular uptake, to enhance the target efficiency, to improve efficacy in binding (e.g., to the targets), to improve patient tolerance to the agent, and/or to reduce toxicity.

### 1) Modifications to Enhance Target Discrimination

In certain embodiments, the RNA silencing agents of the invention may be substituted with a destabilizing nucleotide to enhance single nucleotide target discrimination (see U.S. application Ser. No. 11/698,689, filed Jan. 25, 2007, and U.S. Provisional Application No. 60/762,225 filed Jan. 25, 2006, both of which are incorporated herein by reference). Such a modification may be sufficient to abolish the specificity of the RNA silencing agent for a non-target mRNA (e.g., wild-type mRNA), without appreciably affecting the specificity of the RNA silencing agent for a target mRNA (e.g., gain-of-function mutant mRNA).

In preferred embodiments, the RNA silencing agents of the invention are modified by the introduction of at least one universal nucleotide in the antisense strand thereof. Universal nucleotides comprise base portions that are capable of base pairing indiscriminately with any of the four conventional nucleotide bases (e.g., A, G, C, U). A universal nucleotide is preferred because it has relatively minor effect on the stability of the RNA duplex, or the duplex formed by the guide strand of the RNA silencing agent and the target mRNA. Exemplary universal nucleotide includes those having an inosine base portion or an inosine analog base portion selected from the group consisting of deoxyinosine (e.g., 2′-deoxyinosine), 7-deaza-2′-deoxyinosine, 2′-aza-2′-deoxyinosine, PNA-inosine, morpholino-inosine, LNA-inosine, phosphoramidate-inosine, 2′-O-methoxyethyl-inosine, and 2′-OMe-inosine. In particularly preferred embodiments, the universal nucleotide is an inosine residue or a naturally occurring analog thereof.

In certain embodiments, the RNA silencing agents of the invention are modified by the introduction of at least one destabilizing nucleotide within 5 nucleotides from a specificity-determining nucleotide (i.e., the nucleotide which recognizes the disease-related polymorphism). For example, the destabilizing nucleotide may be introduced at a position that is within 5, 4, 3, 2, or 1 nucleotide(s) from a specificity-determining nucleotide. In exemplary embodiments, the destabilizing nucleotide is introduced at a position which is 3 nucleotides from the specificity-determining nucleotide (i.e., such that there are 2 stabilizing nucleotides between the destabilizing nucleotide and the specificity-determining nucleotide). In RNA silencing agents having two strands or strand portions (e.g., siRNAs and shRNAs), the destabilizing nucleotide may be introduced in the strand or strand portion that does not contain the specificity-determining nucleotide. In preferred embodiments, the destabilizing nucleotide is introduced in the same strand or strand portion that contains the specificity-determining nucleotide.

### 2) Modifications to Enhance Efficacy and Specificity

In certain embodiments, the RNA silencing agents of the invention may be altered to facilitate enhanced efficacy and specificity in mediating RNAi according to asymmetry design rules (see U.S. Pat. Nos. 8,309,704, 7,750,144, 8,304,530, 8,329,892 and 8,309,705). Such alterations facilitate entry of the antisense strand of the siRNA (e.g., a siRNA designed using the methods of the invention or an siRNA produced from a shRNA) into RISC in favor of the sense strand, such that the antisense strand preferentially guides cleavage or translational repression of a target mRNA, and thus increasing or improving the efficiency of target cleavage and silencing. Preferably the asymmetry of an RNA silencing agent is enhanced by lessening the base pair strength between the antisense strand 5′ end (AS 5′) and the sense strand 3′ end (S 3′) of the RNA silencing agent relative to the bond strength or base pair strength between the antisense strand 3′ end (AS 3′) and the sense strand 5′ end (S ′5) of said RNA silencing agent.

In one embodiment, the asymmetry of an RNA silencing agent of the invention may be enhanced such that there are fewer G:C base pairs between the 5′ end of the first or antisense strand and the 3′ end of the sense strand portion than between the 3′ end of the first or antisense strand and the 5′ end of the sense strand portion. In another embodiment, the asymmetry of an RNA silencing agent of the invention may be enhanced such that there is at least one mismatched base pair between the 5′ end of the first or antisense strand and the 3′ end of the sense strand portion. Preferably, the mismatched base pair is selected from the group consisting of G:A, C:A, C:U, G:G, A:A, C:C and U:U. In another embodiment, the asymmetry of an RNA silencing agent of the invention may be enhanced such that there is at least one wobble base pair, e.g., G:U, between the 5′ end of the first or antisense strand and the 3′ end of the sense strand portion. In another embodiment, the asymmetry of an RNA silencing agent of the invention may be enhanced such that there is at least one base pair comprising a rare nucleotide, e.g., inosine (I). Preferably, the base pair is selected from the group consisting of an I:A, I:U and I:C. In yet another embodiment, the asymmetry of an RNA silencing agent of the invention may be enhanced such that there is at least one base pair comprising a modified nucleotide. In preferred embodiments, the modified nucleotide is selected from the group consisting of 2-amino-G, 2-amino-A, 2,6-diamino-G, and 2,6-diamino-A.

3) RNA Silencing Agents with Enhanced Stability

The RNA silencing agents of the present invention can be modified to improve stability in serum or in growth medium for cell cultures. In order to enhance the stability, the 3′-residues may be stabilized against degradation, e.g., they may be selected such that they consist of purine nucleotides, particularly adenosine or guanosine nucleotides. Alternatively, substitution of pyrimidine nucleotides by modified analogues, e.g., substitution of uridine by 2′-deoxythymidine is tolerated and does not affect the efficiency of RNA interference.

In a preferred aspect, the invention features RNA silencing agents that include first and second strands wherein the second strand and/or first strand is modified by the substitution of internal nucleotides with modified nucleotides, such that in vivo stability is enhanced as compared to a corresponding unmodified RNA silencing agent. As defined herein, an “internal” nucleotide is one occurring at any position other than the 5′ end or 3′ end of nucleic acid molecule, polynucleotide or oligonucleotide. An internal nucleotide can be within a single-stranded molecule or within a strand of a duplex or double-stranded molecule. In one embodiment, the sense strand and/or antisense strand is modified by the substitution of at least one internal nucleotide. In another embodiment, the sense strand and/or antisense strand is modified by the substitution of at least 2, 3, 4, 5, 6, 7, 8, 9, 10, 11, 12, 13, 14, 15, 16, 17, 18, 19, 20, 21, 22, 23, 24, 25 or more internal nucleotides. In another embodiment, the sense strand and/or antisense strand is modified by the substitution of at least 5%, 10%, 15%, 20%, 25%, 30%, 35%, 40%, 45%, 50%, 55%, 60%, 65%, 70%, 75%, 80%, 85%, 90%, 95% or more of the internal nucleotides. In yet another embodiment, the sense strand and/or antisense strand is modified by the substitution of all of the internal nucleotides.

In a preferred embodiment of the present invention, the RNA silencing agents may contain at least one modified nucleotide analogue. The nucleotide analogues may be located at positions where the target-specific silencing activity, e.g., the RNAi mediating activity or translational repression activity is not substantially effected, e.g., in a region at the 5′-end and/or the 3′-end of the siRNA molecule. Particularly, the ends may be stabilized by incorporating modified nucleotide analogues.

Exemplary nucleotide analogues include sugar- and/or backbone-modified ribonucleotides (i.e., include modifications to the phosphate-sugar backbone). For example, the phosphodiester linkages of natural RNA may be modified to include at least one of a nitrogen or sulfur heteroatom. In exemplary backbone-modified ribonucleotides, the phosphoester group connecting to adjacent ribonucleotides is replaced by a modified group, e.g., of phosphothioate group. In exemplary sugar-modified ribonucleotides, the 2′ OH-group is replaced by a group selected from H, OR, R, halo, SH, SR, NH₂, NHR, NR₂ or ON, wherein R is C₁-C₆ alkyl, alkenyl or alkynyl and halo is F, Cl, Br or I.

In particular embodiments, the modifications are 2′-fluoro, 2′-amino and/or 2′-thio modifications. Particularly preferred modifications include 2′-fluoro-cytidine, 2′-fluoro-uridine, 2′-fluoro-adenosine, 2′-fluoro-guanosine, 2′-amino-cytidine, 2′-amino-uridine, 2′-amino-adenosine, 2′-amino-guanosine, 2,6-diaminopurine, 4-thio-uridine, and/or 5-amino-allyl-uridine. In a particular embodiment, the 2′-fluoro ribonucleotides are every uridine and cytidine. Additional exemplary modifications include 5-bromo-uridine, 5-iodo-uridine, 5-methyl-cytidine, ribo-thymidine, 2-aminopurine, 2′-amino-butyryl-pyrene-uridine, 5-fluoro-cytidine, and 5-fluoro-uridine. 2′-deoxy-nucleotides and 2′-Ome nucleotides can also be used within modified RNA-silencing agents moities of the instant invention. Additional modified residues include, deoxy-abasic, inosine, N3-methyl-uridine, N6,N6-dimethyl-adenosine, pseudouridine, purine ribonucleoside and ribavirin. In a particularly preferred embodiment, the 2′ moiety is a methyl group such that the linking moiety is a 2′-O-methyl oligonucleotide.

In an exemplary embodiment, the RNA silencing agent of the invention comprises Locked Nucleic Acids (LNAs). LNAs comprise sugar-modified nucleotides that resist nuclease activities (are highly stable) and possess single nucleotide discrimination for mRNA (Elmen et al., Nucleic Acids Res., (2005), 33(1): 439-447; Braasch et al. (2003) Biochemistry 42:7967-7975, Petersen et al. (2003) Trends Biotechnol 21:74-81). These molecules have 2′-O,4′-C-ethylene-bridged nucleic acids, with possible modifications such as 2′-deoxy-2″-fluorouridine. Moreover, LNAs increase the specificity of oligonucleotides by constraining the sugar moiety into the 3′-endo conformation, thereby pre-organizing the nucleotide for base pairing and increasing the melting temperature of the oligonucleotide by as much as 10° C. per base.

In another exemplary embodiment, the RNA silencing agent of the invention comprises Peptide Nucleic Acids (PNAs). PNAs comprise modified nucleotides in which the sugar-phosphate portion of the nucleotide is replaced with a neutral 2-amino ethylglycine moiety capable of forming a polyamide backbone which is highly resistant to nuclease digestion and imparts improved binding specificity to the molecule (Nielsen, et al., Science, (2001), 254: 1497-1500).

Also preferred are nucleobase-modified ribonucleotides, i.e., ribonucleotides, containing at least one non-naturally occurring nucleobase instead of a naturally occurring nucleobase. Bases may be modified to block the activity of adenosine deaminase. Exemplary modified nucleobases include, but are not limited to, uridine and/or cytidine modified at the 5-position, e.g., 5-(2-amino)propyl uridine, 5-bromo uridine; adenosine and/or guanosines modified at the 8 position, e.g., 8-bromo guanosine; deaza nucleotides, e.g., 7-deaza-adenosine; O- and N-alkylated nucleotides, e.g., N6-methyl adenosine are suitable. It should be noted that the above modifications may be combined.

In other embodiments, cross-linking can be employed to alter the pharmacokinetics of the RNA silencing agent, for example, to increase half-life in the body. Thus, the invention includes RNA silencing agents having two complementary strands of nucleic acid, wherein the two strands are crosslinked. The invention also includes RNA silencing agents which are conjugated or unconjugated (e.g., at its 3′ terminus) to another moiety (e.g., a non-nucleic acid moiety such as a peptide), an organic compound (e.g., a dye), or the like). Modifying siRNA derivatives in this way may improve cellular uptake or enhance cellular targeting activities of the resulting siRNA derivative as compared to the corresponding siRNA, are useful for tracing the siRNA derivative in the cell or improve the stability of the siRNA derivative compared to the corresponding siRNA.

Other exemplary modifications include: (a) 2′ modification, e.g., provision of a 2′ OMe moiety on a U in a sense or antisense strand, but especially on a sense strand, or provision of a 2′ OMe moiety in a 3′ overhang, e.g., at the 3′ terminus (3′ terminus means at the 3′ atom of the molecule or at the most 3′ moiety, e.g., the most 3′ P or 2′ position, as indicated by the context); (b) modification of the backbone, e.g., with the replacement of an 0 with an S, in the phosphate backbone, e.g., the provision of a phosphorothioate modification, on the U or the A or both, especially on an antisense strand; e.g., with the replacement of a P with an S; (c) replacement of the U with a C5 amino linker; (d) replacement of an A with a G (sequence changes are preferred to be located on the sense strand and not the antisense strand); and (d) modification at the 2′, 6′, 7, or 8′ position. Exemplary embodiments are those in which one or more of these modifications are present on the sense but not the antisense strand, or embodiments where the antisense strand has fewer of such modifications. Yet other exemplary modifications include the use of a methylated P in a 3′ overhang, e.g., at the 3′ terminus; combination of a 2′ modification, e.g., provision of a 2′ O Me moiety and modification of the backbone, e.g., with the replacement of a P with an S, e.g., the provision of a phosphorothioate modification, or the use of a methylated P, in a 3′ overhang, e.g., at the 3′ terminus; modification with a 3′ alkyl; modification with an abasic pyrrolidone in a 3′ overhang, e.g., at the 3′ terminus; modification with naproxen, ibuprofen, or other moieties which inhibit degradation at the 3′ terminus.

### 4) Modifications to Enhance Cellular Uptake

In other embodiments, RNA silencing agents may be modified with chemical moieties, for example, to enhance cellular uptake by target cells (e.g., neuronal cells). Thus, the invention includes RNA silencing agents which are conjugated or unconjugated (e.g., at its 3′ terminus) to another moiety (e.g., a non-nucleic acid moiety such as a peptide), an organic compound (e.g., a dye), or the like. The conjugation can be accomplished by methods known in the art, e.g., using the methods of Lambert et al., Drug Deliv. Rev.: 47(1), 99-112 (2001) (describes nucleic acids loaded to polyalkylcyanoacrylate (PACA) nanoparticles); Fattal et al., J. Control Release 53(1-3):137-43 (1998) (describes nucleic acids bound to nanoparticles); Schwab et al., Ann. Oncol. 5 Suppl. 4:55-8 (1994) (describes nucleic acids linked to intercalating agents, hydrophobic groups, polycations or PACA nanoparticles); and Godard et al., Eur. J. Biochem. 232(2):404-10 (1995) (describes nucleic acids linked to nanoparticles).

In a particular embodiment, an RNA silencing agent of invention is conjugated to a lipophilic moiety. In one embodiment, the lipophilic moiety is a ligand that includes a cationic group. In another embodiment, the lipophilic moiety is attached to one or both strands of an siRNA. In an exemplary embodiment, the lipophilic moiety is attached to one end of the sense strand of the siRNA. In another exemplary embodiment, the lipophilic moiety is attached to the Y end of the sense strand. In certain embodiments, the lipophilic moiety is selected from the group consisting of cholesterol, vitamin E, vitamin K, vitamin A, folic acid, or a cationic dye (e.g., Cy3). In an exemplary embodiment, the lipophilic moiety is a cholesterol. Other lipophilic moieties include cholic acid, adamantane acetic acid, 1-pyrene butyric acid, dihydrotestosterone, 1,3-Bis-O(hexadecyl)glycerol, geranyloxyhexyl group, hexadecylglycerol, borneol, menthol, 1,3-propanediol, heptadecyl group, palmitic acid, myristic acid, 03-(oleoyl)lithocholic acid, 03-(oleoyl)cholenic acid, dimethoxytrityl, or phenoxazine.

### 5) Tethered Ligands

Other entities can be tethered to an RNA silencing agent of the invention. For example, a ligand tethered to an RNA silencing agent to improve stability, hybridization thermodynamics with a target nucleic acid, targeting to a particular tissue or cell-type, or cell permeability, e.g., by an endocytosis-dependent or -independent mechanism. Ligands and associated modifications can also increase sequence specificity and consequently decrease off-site targeting. A tethered ligand can include one or more modified bases or sugars that can function as intercalators. These are preferably located in an internal region, such as in a bulge of RNA silencing agent/target duplex. The intercalator can be an aromatic, e.g., a polycyclic aromatic or heterocyclic aromatic compound. A polycyclic intercalator can have stacking capabilities, and can include systems with 2, 3, or 4 fused rings. The universal bases described herein can be included on a ligand. In one embodiment, the ligand can include a cleaving group that contributes to target gene inhibition by cleavage of the target nucleic acid. The cleaving group can be, for example, a bleomycin (e.g., bleomycin-A5, bleomycin-A2, or bleomycin-B2), pyrene, phenanthroline (e.g., 0-phenanthroline), a polyamine, a tripeptide (e.g., lys-tyr-lys tripeptide), or metal ion chelating group. The metal ion chelating group can include, e.g., an Lu(III) or EU(III) macrocyclic complex, a Zn(II) 2,9-dimethylphenanthroline derivative, a Cu(II) terpyridine, or acridine, which can promote the selective cleavage of target RNA at the site of the bulge by free metal ions, such as Lu(III). In some embodiments, a peptide ligand can be tethered to an RNA silencing agent to promote cleavage of the target RNA, e.g., at the bulge region. For example, 1,8-dimethyl-1,3,6,8,10,13-hexaazacyclotetradecane (cyclam) can be conjugated to a peptide (e.g., by an amino acid derivative) to promote target RNA cleavage. A tethered ligand can be an aminoglycoside ligand, which can cause an RNA silencing agent to have improved hybridization properties or improved sequence specificity. Exemplary aminoglycosides include glycosylated polylysine, galactosylated polylysine, neomycin B, tobramycin, kanamycin A, and acridine conjugates of aminoglycosides, such as Neo-N-acridine, Neo-S-acridine, Neo-C-acridine, Tobra-N-acridine, and KanaA-N-acridine. Use of an acridine analog can increase sequence specificity. For example, neomycin B has a high affinity for RNA as compared to DNA, but low sequence-specificity. An acridine analog, neo-5-acridine has an increased affinity for the HIV Rev-response element (RRE). In some embodiments the guanidine analog (the guanidinoglycoside) of an aminoglycoside ligand is tethered to an RNA silencing agent. In a guanidinoglycoside, the amine group on the amino acid is exchanged for a guanidine group. Attachment of a guanidine analog can enhance cell permeability of an RNA silencing agent. A tethered ligand can be a poly-arginine peptide, peptoid or peptidomimetic, which can enhance the cellular uptake of an oligonucleotide agent.

Exemplary ligands are coupled, preferably covalently, either directly or indirectly via an intervening tether, to a ligand-conjugated carrier. In exemplary embodiments, the ligand is attached to the carrier via an intervening tether. In exemplary embodiments, a ligand alters the distribution, targeting or lifetime of an RNA silencing agent into which it is incorporated. In exemplary embodiments, a ligand provides an enhanced affinity for a selected target, e.g., molecule, cell or cell type, compartment, e.g., a cellular or organ compartment, tissue, organ or region of the body, as, e.g., compared to a species absent such a ligand.

Exemplary ligands can improve transport, hybridization, and specificity properties and may also improve nuclease resistance of the resultant natural or modified RNA silencing agent, or a polymeric molecule comprising any combination of monomers described herein and/or natural or modified ribonucleotides. Ligands in general can include therapeutic modifiers, e.g., for enhancing uptake; diagnostic compounds or reporter groups e.g., for monitoring distribution; cross-linking agents; nuclease-resistance conferring moieties; and natural or unusual nucleobases. General examples include lipophiles, lipids, steroids (e.g., uvaol, hecigenin, diosgenin), terpenes (e.g., triterpenes, e.g., sarsasapogenin, Friedelin, epifriedelanol derivatized lithocholic acid), vitamins (e.g., folic acid, vitamin A, biotin, pyridoxal), carbohydrates, proteins, protein binding agents, integrin targeting molecules, polycationics, peptides, polyamines, and peptide mimics. Ligands can include a naturally occurring substance, (e.g., human serum albumin (HSA), low-density lipoprotein (LDL), or globulin); carbohydrate (e.g., a dextran, pullulan, chitin, chitosan, inulin, cyclodextrin or hyaluronic acid); amino acid, or a lipid. The ligand may also be a recombinant or synthetic molecule, such as a synthetic polymer, e.g., a synthetic polyamino acid. Examples of polyamino acids include polyamino acid is a polylysine (PLL), poly L-aspartic acid, poly L-glutamic acid, styrene-maleic acid anhydride copolymer, poly(L-lactide-co-glycolied) copolymer, divinyl ether-maleic anhydride copolymer, N-(2-hydroxypropyl)methacrylamide copolymer (HMPA), polyethylene glycol (PEG), polyvinyl alcohol (PVA), polyurethane, poly(2-ethylacryllic acid), N-isopropylacrylamide polymers, or polyphosphazine. Example of polyamines include: polyethylenimine, polylysine (PLL), spermine, spermidine, polyamine, pseudopeptide-polyamine, peptidomimetic polyamine, dendrimer polyamine, arginine, amidine, protamine, cationic lipid, cationic porphyrin, quaternary salt of a polyamine, or an alpha helical peptide.

Ligands can also include targeting groups, e.g., a cell or tissue targeting agent, e.g., a lectin, glycoprotein, lipid or protein, e.g., an antibody, that binds to a specified cell type such as a kidney, gland (e.g., thyroid gland), brain, eye, and/or male testis cell. A targeting group can be a thyrotropin, melanotropin, lectin, glycoprotein, surfactant protein A, mucin carbohydrate, multivalent lactose, multivalent galactose, N-acetyl-galactosamine, N-acetyl-glucosamine, multivalent mannose, multivalent fucose, glycosylated polyaminoacids, multivalent galactose, transferrin, bisphosphonate, polyglutamate, polyaspartate, a lipid, cholesterol, a steroid, bile acid, folate, vitamin B12, biotin, or an RGD peptide or RGD peptide mimetic. Other examples of ligands include dyes, intercalating agents (e.g. acridines and substituted acridines), cross-linkers (e.g. psoralene, mitomycin C), porphyrins (TPPC4, texaphyrin, Sapphyrin), polycyclic aromatic hydrocarbons (e.g., phenazine, dihydrophenazine, phenanthroline, pyrenes), lys-tyr-lys tripeptide, aminoglycosides, guanidium aminoglycodies, artificial endonucleases (e.g. EDTA), lipophilic molecules, e.g., cholesterol (and thio analogs thereof), cholic acid, cholanic acid, lithocholic acid, adamantane acetic acid, 1-pyrene butyric acid, dihydrotestosterone, glycerol (e.g., esters (e.g., mono, bis, or tris fatty acid esters, e.g., C₁₀, C₁₁, C₁₂, C₁₃, C₁₄, C₁₅, C₁₆, C₁₇, C₁₈, C₁₉, or C₂₀ fatty acids) and ethers thereof, e.g., C₁₀, C₁₁, C₁₂, C₁₃, C₁₄, C₁₅, C₁₆, C₁₇, C₁₈, C₁₉, or C₂₀ alkyl; e.g., 1,3-bis-O(hexadecyl)glycerol, 1,3-bis-O(octaadecyl)glycerol), geranyloxyhexyl group, hexadecylglycerol, borneol, menthol, 1,3-propanediol, heptadecyl group, palmitic acid, stearic acid (e.g., glyceryl distearate), oleic acid, myristic acid, O3-(oleoyl)lithocholic acid, O3-(oleoyl)cholenic acid, dimethoxytrityl, or phenoxazine) and peptide conjugates (e.g., antennapedia peptide, Tat peptide), alkylating agents, phosphate, amino, mercapto, PEG (e.g., PEG-40K), MPEG, [MPEG]₂, polyamino, alkyl, substituted alkyl, radiolabeled markers, enzymes, haptens (e.g. biotin), transport/absorption facilitators (e.g., aspirin, naproxen, vitamin E, folic acid), synthetic ribonucleases (e.g., imidazole, bisimidazole, histamine, imidazole clusters, acridine-imidazole conjugates, Eu3+ complexes of tetraazamacrocycles), dinitrophenyl, HRP or AP.

Ligands can be proteins, e.g., glycoproteins, or peptides, e.g., molecules having a specific affinity for a co-ligand, or antibodies e.g., an antibody, that binds to a specified cell type such as a cancer cell, endothelial cell, or bone cell. Ligands may also include hormones and hormone receptors. They can also include non-peptidic species, such as lipids, lectins, carbohydrates, vitamins, cofactors, multivalent lactose, multivalent galactose, N-acetyl-galactosamine, N-acetyl-glucosamine multivalent mannose, or multivalent fucose. The ligand can be, for example, a lipopolysaccharide, an activator of p38 MAP kinase, or an activator of NF-κB.

The ligand can be a substance, e.g., a drug, which can increase the uptake of the RNA silencing agent into the cell, for example, by disrupting the cell's cytoskeleton, e.g., by disrupting the cell's microtubules, microfilaments, and/or intermediate filaments. The drug can be, for example, taxon, vincristine, vinblastine, cytochalasin, nocodazole, japlakinolide, latrunculin A, phalloidin, swinholide A, indanocine, or myoservin. The ligand can increase the uptake of the RNA silencing agent into the cell by activating an inflammatory response, for example. Exemplary ligands that would have such an effect include tumor necrosis factor alpha (TNFα), interleukin-1 beta, or gamma interferon. In one aspect, the ligand is a lipid or lipid-based molecule. Such a lipid or lipid-based molecule preferably binds a serum protein, e.g., human serum albumin (HSA). An HSA binding ligand allows for distribution of the conjugate to a target tissue, e.g., a non-kidney target tissue of the body. For example, the target tissue can be the liver, including parenchymal cells of the liver. Other molecules that can bind HSA can also be used as ligands. For example, neproxin or aspirin can be used. A lipid or lipid-based ligand can (a) increase resistance to degradation of the conjugate, (b) increase targeting or transport into a target cell or cell membrane, and/or (c) can be used to adjust binding to a serum protein, e.g., HSA. A lipid-based ligand can be used to modulate, e.g., control the binding of the conjugate to a target tissue. For example, a lipid or lipid-based ligand that binds to HSA more strongly will be less likely to be targeted to the kidney and therefore less likely to be cleared from the body. A lipid or lipid-based ligand that binds to HSA less strongly can be used to target the conjugate to the kidney. In a preferred embodiment, the lipid-based ligand binds HSA. A lipid-based ligand can bind HSA with a sufficient affinity such that the conjugate will be preferably distributed to a non-kidney tissue. However, it is preferred that the affinity not be so strong that the HSA-ligand binding cannot be reversed. In another preferred embodiment, the lipid-based ligand binds HSA weakly or not at all, such that the conjugate will be preferably distributed to the kidney. Other moieties that target to kidney cells can also be used in place of or in addition to the lipid-based ligand.

In another aspect, the ligand is a moiety, e.g., a vitamin, which is taken up by a target cell, e.g., a proliferating cell. These are particularly useful for treating disorders characterized by unwanted cell proliferation, e.g., of the malignant or non-malignant type, e.g., cancer cells. Exemplary vitamins include vitamin A, E, and K. Other exemplary vitamins include are B vitamin, e.g., folic acid, B12, riboflavin, biotin, pyridoxal or other vitamins or nutrients taken up by cancer cells. Also included are HSA and low-density lipoprotein (LDL).

In another aspect, the ligand is a cell-permeation agent, preferably a helical cell-permeation agent. Preferably, the agent is amphipathic. An exemplary agent is a peptide such as tat or antennopedia. If the agent is a peptide, it can be modified, including a peptidylmimetic, invertomers, non-peptide or pseudo-peptide linkages, and use of D-amino acids. The helical agent is preferably an alpha-helical agent, which preferably has a lipophilic and a lipophobic phase.

The ligand can be a peptide or peptidomimetic. A peptidomimetic (also referred to herein as an oligopeptidomimetic) is a molecule capable of folding into a defined three-dimensional structure similar to a natural peptide. The attachment of peptide and peptidomimetics to oligonucleotide agents can affect pharmacokinetic distribution of the RNA silencing agent, such as by enhancing cellular recognition and absorption. The peptide or peptidomimetic moiety can be about 5-50 amino acids long, e.g., about 5, 10, 15, 20, 25, 30, 35, 40, 45, or 50 amino acids long. A peptide or peptidomimetic can be, for example, a cell permeation peptide, cationic peptide, amphipathic peptide, or hydrophobic peptide (e.g., consisting primarily of Tyr, Trp or Phe). The peptide moiety can be a dendrimer peptide, constrained peptide or crosslinked peptide. The peptide moiety can be an L-peptide or D-peptide. In another alternative, the peptide moiety can include a hydrophobic membrane translocation sequence (MTS). A peptide or peptidomimetic can be encoded by a random sequence of DNA, such as a peptide identified from a phage-display library, or one-bead-one-compound (OBOC) combinatorial library (Lam et al., Nature 354:82-84, 1991). In exemplary embodiments, the peptide or peptidomimetic tethered to an RNA silencing agent via an incorporated monomer unit is a cell targeting peptide such as an arginine-glycine-aspartic acid (RGD)-peptide, or RGD mimic. A peptide moiety can range in length from about 5 amino acids to about 40 amino acids. The peptide moieties can have a structural modification, such as to increase stability or direct conformational properties. Any of the structural modifications described below can be utilized.

### Oligonucleotide Conjugate

The oligonucleotide conjugates described herein comprise an oligonucleotide and a dendron. The dendron can have a strong reversible and non-covalent albumin binding that can be advantageously used to minimize degradation, reduce their uptake and degradation by macrophages, prevent non-specific uptake by cells, and/or provide enhanced delivery to a tumor of the oligonucleotide conjugate. The dendron can also have a nanomolar affinity to albumin that advantageously ensures that the albumin-oligonucleotide conjugate complex remains stable throughout biodistribution, cell interactions, and/or treatment.

In some embodiments, the oligonucleotide conjugate has a structure of Formula I:

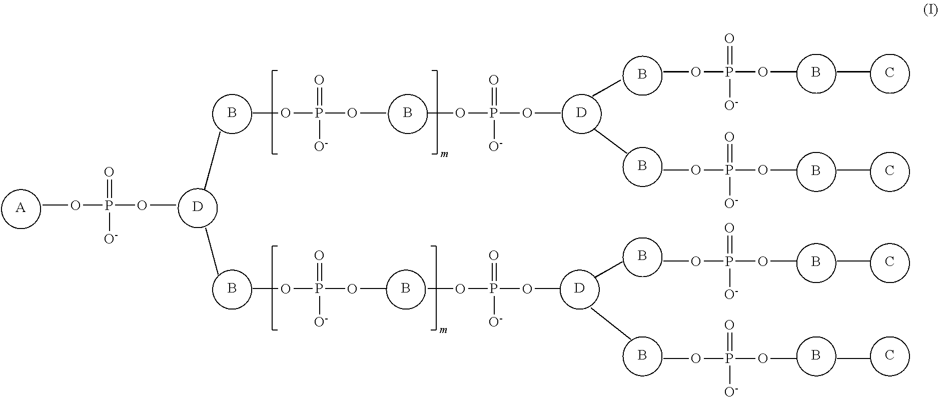

wherein:
A is an oligonucleotide;
B, for each occurrence, independently comprises one or more hydrophobic chains, amines, amides, esters, N- or O-containing heterocycles, thioethers, disulfides, and/or aromatic rings, wherein the hydrophobic chain comprises a saturated or unsaturated C₁₋₂₄ alkyl chain;
C, for each occurrence, independently comprises: an hydrophilic group comprising hydroxide, amine, phosphate, sulfur, and/or sugar; an hydrophobic group comprising amine, amide, ether, ester, N- or O-containing heterocycles, thiol, thioether, and/or saturated or unsaturated C₁₋₂₄ alkyl chain; and/or one or more aromatic rings;
D, for each occurrence, independently is a branching unit comprising one or more alkyl chains, amides, ethers, esters, and amines, wherein the branching unit comprises 2 to 4 branches; and
m, for each occurrence, independently is 0 or 1.

In some embodiments, the oligonucleotide conjugate has a structure of Formula II:

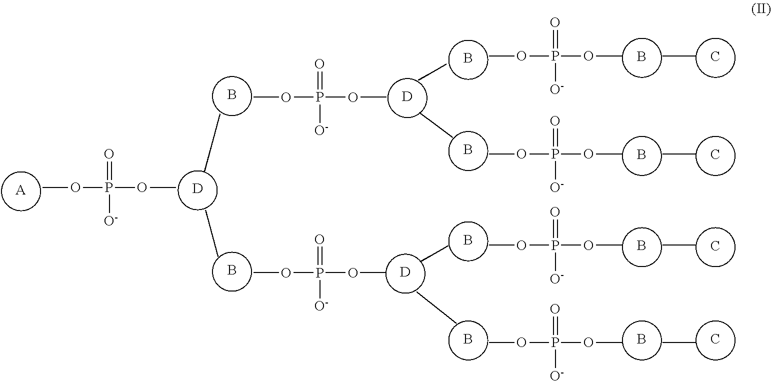

wherein:
A is an oligonucleotide;
B, for each occurrence, independently comprises one or more hydrophobic chains, amines, amides, esters, N- or O-containing heterocycles, thioethers, disulfides, and/or aromatic rings, wherein the hydrophobic chain comprises a saturated or unsaturated C₁₋₂₄ alkyl chain;
C, for each occurrence, independently comprises: an hydrophilic group comprising hydroxide, amine, phosphate, sulfur, and/or sugar; an hydrophobic group comprising amine, amide, ether, ester, N- or O-containing heterocycles, thiol, thioether, and/or saturated or unsaturated C₁₋₂₄ alkyl chain; and/or one or more aromatic rings; and
D, for each occurrence, independently is a branching unit comprising one or more alkyl chains, amides, ethers, esters, and amines, wherein the branching unit comprises 2 to 4 branches.

In some embodiments, the oligonucleotide conjugate has a structure of Formula III:

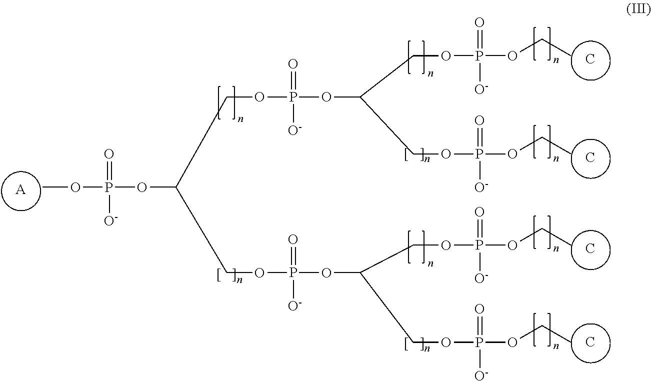

wherein n, for each occurrence, independently is an integer between 1-24. In some embodiments, C is OH and n, for each occurrence, independently is 1, 6, or 12. In some embodiments, the oligonucleotide conjugate has the structure of Formula IV:

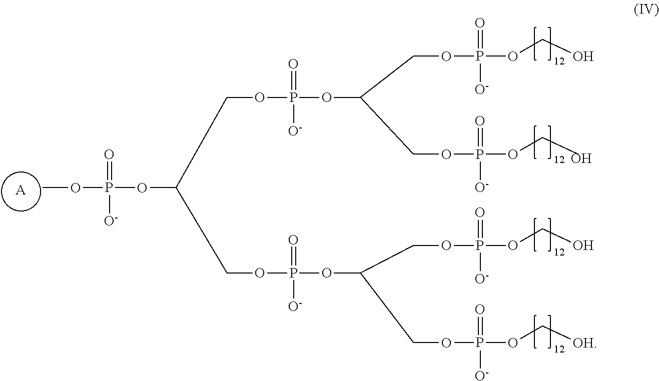

In some embodiments, the oligonucleotide conjugate has a structure of Formula V:

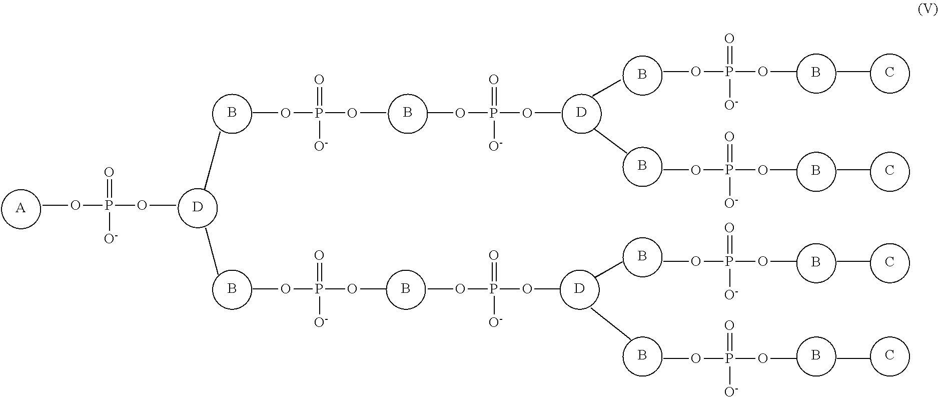

wherein:
A is an oligonucleotide;
B, for each occurrence, independently comprises one or more hydrophobic chains, amines, amides, esters, N- or O-containing heterocycles, thioethers, disulfides, and/or aromatic rings, wherein the hydrophobic chain comprises a saturated or unsaturated C₁₋₂₄ alkyl chain;
C, for each occurrence, independently comprises: an hydrophilic group comprising hydroxide, amine, phosphate, sulfur, and/or sugar; an hydrophobic group comprising amine, amide, ether, ester, N- or O-containing heterocycles, thiol, thioether, and/or saturated or unsaturated C₁₋₂₄ alkyl chain; and/or one or more aromatic rings; and
D, for each occurrence, independently is a branching unit comprising one or more alkyl chains, amides, ethers, esters, and amines, wherein the branching unit comprises 2 to 4 branches. In some embodiments, the oligonucleotide conjugate has a structure of Formula VI:

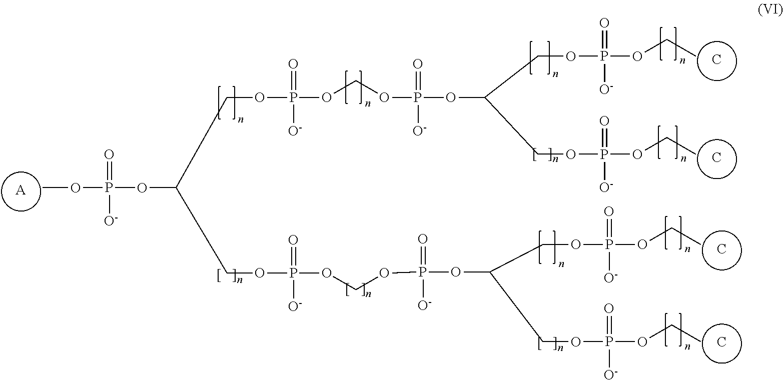

wherein n, for each occurrence, independently is an integer between 1-24. In some embodiments, C, for each occurrence, independently comprises: an hydrophilic group comprising hydroxide, amine, phosphate, sulfur, and/or sugar; an hydrophobic group comprising amine, amide, ether, ester, N- or O-containing heterocycles, thiol, thioether, and/or saturated or unsaturated C₁₋₂₄ alkyl chain; and/or one or more aromatic rings. In some embodiments, C is OH. In some embodiments, n, for each occurrence, independently is 1, 6, or 12.

In some embodiments, the oligonucleotide conjugate has the structure of Formula VII:

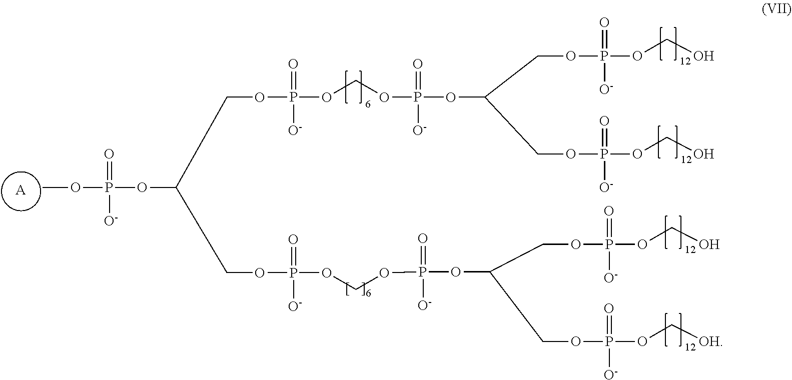

### Branched Oligonucleotide Conjugate

In certain embodiments, the oligonucleotide conjugate is a branched oligonucleotide conjugate.

The branched oligonucleotides conjugate described here comprise two or more glucosamines or derivatives thereof that are linked together. The different branched oligonucleotides conjugates described herein (e.g., a branched oligonucleotide conjugate with two, three, or four glucosamine or derivative thereof) can enhance delivery of the oligonucleotide, including kidney, gland (e.g., thyroid gland), brain, eye, and male testis cell-specific delivery.

In certain embodiments, the branched oligonucleotide conjugate comprises: i) an oligonucleotide comprising a 5′ and a 3′ end and complementarity to a target nucleic acid; and ii) a branched functional moiety that is linked to the oligonucleotide and comprises two or more glucosamines or derivatives thereof.

In certain embodiments, the branched functional moiety is a triple amine functional moiety such as a phosphatidylcholine (PC) esterified triple amine (PC-triple amine).

In certain embodiments, the two or more glucosamines or derivatives thereof in the branched oligonucleotide conjugate are connected to one another by one or more branched functional moieties independently selected from a linker, a spacer, and a branching point.

In certain embodiments, the linker comprises an ethylene glycol chain, an alkyl chain, a peptide, an RNA, a DNA, a phosphate, a phosphonate, a phosphoramidate, an ester, an amide, a triazole, or any combinations thereof.

In certain embodiments, the branching point comprises a polyvalent organic species or derivative thereof.

In another embodiment, the branching point is an amino acid derivative. In another embodiment of the branching point is selected from the formulas of:

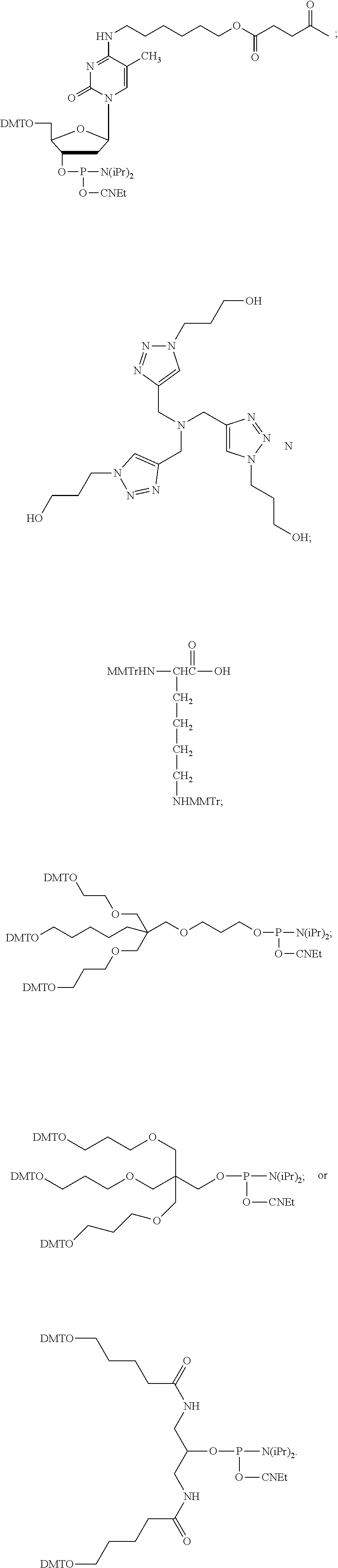

Polyvalent organic species are moieties comprising carbon and three or more valencies (i.e., points of attachment with moieties such as S, L or N, as defined above). Non-limiting examples of polyvalent organic species include triols (e.g., glycerol, phloroglucinol, and the like), tetrols (e.g., ribose, pentaerythritol, 1,2,3,5-tetrahydroxybenzene, and the like), tri-carboxylic acids (e.g., citric acid, 1,3,5-cyclohexanetricarboxylic acid, trimesic acid, and the like), tetra-carboxylic acids (e.g., ethylenediaminetetraacetic acid, pyromellitic acid, and the like), tertiary amines (e.g., tripropargylamine, triethanolamine, and the like), triamines (e.g., diethylenetriamine and the like), tetramines, and species comprising a combination of hydroxyl, thiol, amino, and/or carboxyl moieties (e.g., amino acids such as lysine, serine, cysteine, and the like).

In certain embodiments, the spacer comprises an ethylene glycol chain, an alkyl chain, a peptide, an RNA, a DNA, a phosphate, a phosphonate, a phosphoramidate, an ester, an amide, a triazole, or a combination thereof.

### AIM2-Targeting Oligonucleotide Conjugates

In one aspect, provided herein is an oligonucleotide conjugate comprising i) an oligonucleotide comprising a 5′ and a 3′ end and complementarity to AIM2; and ii) a dendron that is linked to the oligonucleotide and comprises an end group, a phosphate group, and/or a hydrophobic chain.

AIM2 (also known as “absent in melanoma 2” and “interferon-inducible protein AIM2), is a protein that in humans is encoded by the AIM2 gene. AIM2 is involved in the innate immune response and recognizes cytosolic double-stranded DNA. AIM2 is a component of the AIM2 inflammasome, which produces mature IL-10 and IL-18, as well as induces a lytic form of cell death called pyroptosis. AIM2 has been reported to suppress the cGAS-STING-type I IFN signaling axis in bone marrow derived dendritic cells (BMDCs) and macrophages (BMDMs) in response to tumor-derived cytosolic DNA in vitro. An exemplary amino acid sequence of human AIM2 is provided in GenBank Accession No. NP_004824.

In certain embodiments, the oligonucleotide conjugate targeting AIM2 is combined with an immune checkpoint inhibitor (e.g., an inhibitor of PD-1, PD-L1, or CTLA-4) for the treatment of cancer (e.g., melanoma).

US20220175816A1, incorporated herein by reference, describes the inhibition of AIM2, including specific exemplary oligonucleotides targeting AIM2 and the combination with immune checkpoint inhibitors (e.g., anti-PD-1 antibodies).

### Pharmaceutical Compositions and Methods of Administration

In one aspect, provided herein is a pharmaceutical composition comprising a therapeutically effective amount of one or more oligonucleotide conjugates as described herein, and a pharmaceutically acceptable carrier.

A pharmaceutical composition of the invention is formulated to be compatible with its intended route of administration. Examples of routes of administration include parenteral, e.g., intravenous (IV), intradermal, subcutaneous (SC or SQ), intraperitoneal, intramuscular, oral (e.g., inhalation), transdermal (topical), and transmucosal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

Compositions, such as pharmaceutical compositions, methods, and systems comprising the oligonucleotide conjugate provided herein can encompass any pharmaceutically acceptable salts, esters, or salts of such esters. The disclosure herein is also drawn to pharmaceutically acceptable salts of oligonucleotide conjugates, prodrugs, pharmaceutically acceptable salts of such prodrugs, and other bioequivalents. Suitable pharmaceutically acceptable salts include, but are not limited to, sodium and potassium salts. In some embodiments, the disclosure provides salts of the oligonucleotide conjugates. In some embodiments, the salt is a sodium salt. In some embodiments, the salt is a potassium salt.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the EC50 (i.e., the concentration of the test compound which achieves a half-maximal response) as determined in cell culture. Such information can be used to more accurately determine useful doses in humans. Levels in plasma may be measured, for example, by high performance liquid chromatography.

### Kits

In certain other aspects, the invention provides kits that include a suitable container containing a pharmaceutical formulation of an RNA silencing agent, e.g., a double-stranded RNA silencing agent, or sRNA agent, (e.g., a precursor, e.g., a larger RNA silencing agent which can be processed into a sRNA agent, or a DNA which encodes an RNA silencing agent, e.g., a double-stranded RNA silencing agent, or sRNA agent, or precursor thereof). In certain embodiments the individual components of the pharmaceutical formulation may be provided in one container. Alternatively, it may be desirable to provide the components of the pharmaceutical formulation separately in two or more containers, e.g., one container for an RNA silencing agent preparation, and at least another for a carrier compound. The kit may be packaged in a number of different configurations such as one or more containers in a single box. The different components can be combined, e.g., according to instructions provided with the kit. The components can be combined according to a method described herein, e.g., to prepare and administer a pharmaceutical composition. The kit can also include a delivery device.

### Methods of Treatment

In one aspect, provided herein is a method for selectively delivering one or more oligonucleotide conjugates as described herein, to the tumor of in a patient, comprising administering said compound to the patient.

In certain aspects, provided herein is a method for treating cancer in a patient in need of such treatment, comprising administering to the patient a compound of Formula (I). Non-limiting examples of cancer include: melanoma cancer, brain cancer, breast cancer, cervical cancer, colorectal cancer, eye cancer, fallopian tube cancer, testicular cancer, leukemia cancer, liver cancer, lung cancer, ovarian cancer, pancreatic cancer, prostate cancer, pulmonary cancer, rectal cancer, renal cancer, skin cancer, stomach cancer, and thyroid cancer. In some embodiments, the cancer is associated with the AIM2 gene.

“Treatment,” or “treating,” as used herein, is defined as the application or administration of a therapeutic agent (e.g., a RNA agent or vector or transgene encoding same) to a patient, or application or administration of a therapeutic agent to an isolated tissue or cell line from a patient, who has the disease or disorder, a symptom of disease or disorder or a predisposition toward a disease or disorder, with the purpose to cure, heal, alleviate, relieve, alter, remedy, ameliorate, improve or affect the disease or disorder, the symptoms of the disease or disorder, or the predisposition toward disease.

In one aspect, the invention provides a method for preventing in a subject, a disease or disorder as described above, by administering to the subject a therapeutic agent (e.g., an RNAi agent or vector or transgene encoding same). Subjects at risk for the disease can be identified by, for example, any or a combination of diagnostic or prognostic assays as described herein. Administration of a prophylactic agent can occur prior to the manifestation of symptoms characteristic of the disease or disorder, such that the disease or disorder is prevented or, alternatively, delayed in its progression.

Another aspect of the invention pertains to methods treating subjects therapeutically, i.e., alter onset of symptoms of the disease or disorder. In an exemplary embodiment, the modulatory method of the invention involves contacting a cell expressing a gain-of-function mutant with a therapeutic agent (e.g., a RNAi agent or vector or transgene encoding same) that is specific for one or more target sequences within the gene, such that sequence specific interference with the gene is achieved. These methods can be performed in vitro (e.g., by culturing the cell with the agent) or, alternatively, in vivo (e.g., by administering the agent to a subject).

In one embodiment, a subject is administered an initial dose, and one or more maintenance doses of an RNA silencing agent. The maintenance dose or doses are generally lower than the initial dose, e.g., one-half less of the initial dose. A maintenance regimen can include treating the subject with a dose or doses ranging from 0.01 to 1.4 mg/kg of body weight per day, e.g., 10, 1, 0.1, 0.01, 0.001, or 0.00001 mg per kg of bodyweight per day. The maintenance doses are preferably administered no more than once every 5, 10, or 30 days. Further, the treatment regimen may last for a period of time which will vary depending upon the nature of the particular disease, its severity and the overall condition of the patient. In preferred embodiments the dosage may be delivered no more than once per day, e.g., no more than once per 24, 36, 48, or more hours, e.g., no more than once every 5 or 8 days. Following treatment, the patient can be monitored for changes in his condition and for alleviation of the symptoms of the disease state. The dosage of the compound may either be increased in the event the patient does not respond significantly to current dosage levels, or the dose may be decreased if an alleviation of the symptoms of the disease state is observed, if the disease state has been ablated, or if undesired side-effects are observed.

### EXAMPLES

While several experimental Examples are contemplated, these Examples are intended to be non-limiting.

### Example 1. Oligonucleotide Synthesis

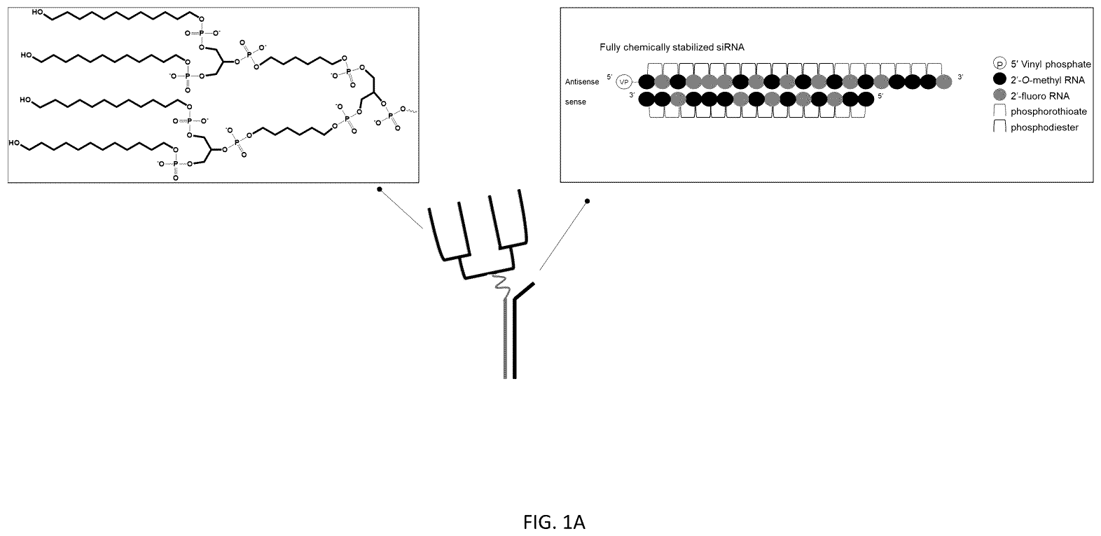

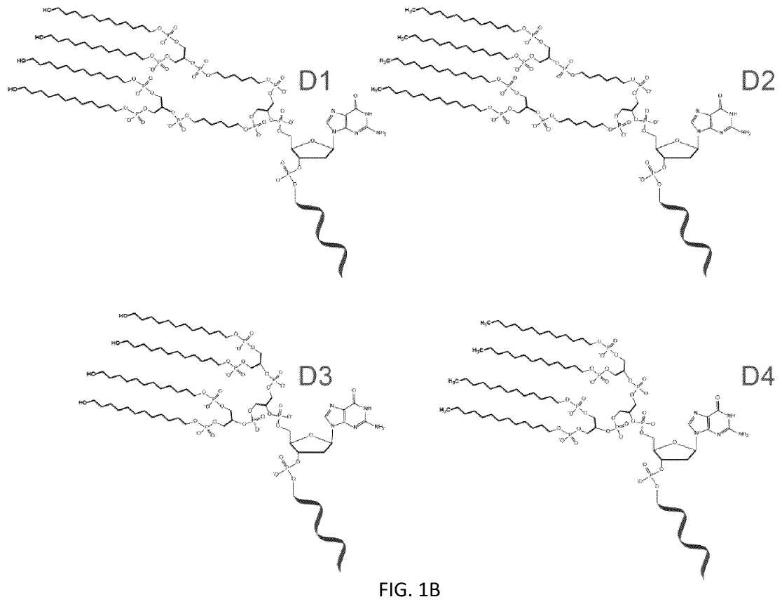

Oligonucleotides were synthesized on a MerMade 6/12 synthesizer (Bioautomation) and AKTA Oligopilot 100 (GE Healthcare Life Sciences) following standard protocols. In brief, conjugated sense strands were synthesized at 5-20 μmol scales on custom-synthesized lipid-functionalized controlled pore glass (CPG) supports for DCA conjugate. For the dendritic sense strand, synthesis was on a CPG functionalized with UnyLinker (ChemGenes) and commercially available amidites (Cy3, C6, C12, and symmetrical branching from ChemGenes and Glen Research) were used to build the dendritic moiety on the 5′-end. All sense strands had a 2dT spacer in between the strand and the conjugate. Antisense strands were synthesized on CPG functionalized with UnyLinker. They were first deprotected with a solution of bromotrimethylsilane/pyridine (3:2, v/v) in dichloromethane for the (E)-vinylphosphonate deprotection, then cleaved and deprotected with 28% aqueous ammonium hydroxide solution for 20 hours at 60° C. All strands were cleaved and deprotected using 28% aqueous ammonium hydroxide solution for 20 hours at 60° C., followed by drying under vacuum at 60° C., and resuspended in Millipore H2O. Oligonucleotides were purified using an Agilent Prostar System (Agilent Technologies) over a C18 column for lipid-conjugated sense strands and over an ion-exchange column for antisense strands. Purified oligonucleotides were desalted by size-exclusion chromatography and characterized by liquid chromatography-mass spectrometry (LCMS) analysis on an Agilent 6530 accurate-mass quadrupole time-of-flight (Q-TOF) LCMS (Agilent Technologies). FIGS. 1A-1B show an example of a fully chemically stabilized oligonucleotide (siRNA) and a dendritic (D) siRNA synthesized using this method.

Sequences of compounds and their modifications are shown in Table 1 below (#t: PS backbone, m: 2′-o-methyl, f: 2′-fluoro, C12: hexaethylene spacer, C6: triethylene spacer, SB: symmetrical branching, V: (E)-vinylphosphonate, D: an exemplary dendrimer conjugate of the instant disclosure, DCA: docosanoic acid conjugate).

TABLE 1
Oligonucleotide sequences synthesized.

| SEQ | | |
|---|---|---|
| ID NO | Strands | Sequences (5′ to 3′) |
| 1 | D-sense (HTT | (C12)(SB)(C6)(SB)(dT)(dT)(mC)#(mA)#(mG)(mU)(fA) |
| | targeting) | (fA)(fA)(mG)(fA)(mG)(mA)(mU)(mU)#(mA)#(mA) |
| 2 | DCA-sense (HTT | (mC)#(mA)#(mG)(mU)(fA)(fA)(fA)(mG)(fA)(mG)(mA) |
| | targeting) | (mU)(mU)#(mA)#(mA)(dT)(dT)-DCA |
| 3 | Unconjugated sense | (mC)#(mA)#(mG)(mU)(fA)(fA)(fA)(mG)(fA)(mG)(mA) |
| | (HTT targeting) | (mU)(mU)#(mA)#(mA)(dT)(dT) |
| 4 | Antisense (HTT | V(mU)#(fU)#(mA)(mA)(mU)(fC)(mU)(mC)(mU)(mU)(mU) |
| | targeting) | (mA)(mC)#(fU)#(mG)#(fA)#(mU)#(mA)#(mU)#(fA) |
| 5 | D-sense (protein | (C12)(SB)(C6)(SB)(dT)(dT)(Cy3)#(mG)#(mU)#(mA)(fC) |
| | binding experiment) | (mA)(fA)(mA)(fG)(mG)(fA)(mA)(mU)(mC)(fU)#(mG)#(mA) |
| 6 | DCA-sense (protein | (Cy3)#(mG)#(mU)#(mA)(fC)(mA)(fA)(mA)(fG)(mG)(fA) |
| | binding experiment) | (mA)(mU)(mC)(fU)#(mG)#(mA)(dT)(dT)-DCA |
| 7 | Unconjugated Sense | (Cy3)#(mG)#(mU)#(mA)(fC)(mA)(fA)(mA)(fG)(mG)(fA) |
| | (protein binding | (mA)(mU)(mC)(fU)#(mG)#(mA)(dT)(dT) |
| | experiment) | |
| 8 | Antisense (protein | V(mU)#(fC)#(mA)(fG)(fA)(fU)(mU)(fC)(mC)(fU)(mU) |
| | binding experiment) | (fU)(mG)(fU)#(mA)#(fC)#(mU)#(mU)#(mC)#(fA)#(mU) |
| 9 | D-sense (AIM2 | (C12)(SB)(C6)(SB)(dT)(dT)(Cy3)#(mC)#(mU)#(mU)(fU) |
| | targeting) | (mG)(fU)(mA)(fA)(mA)(fA)(mG)(mU)(mU)(fU)#(mU)#(mA) |
| 10 | Sense (AIM2 targeting) | (dT)(dT)(mC)#(mU)#(mU)(fU)(mG)(fU)(mA)(fA)(mA) |
| | | (fA)(mG)(mU)(mU)(fU)#(mU)#(mA) |
| 11 | Antisense (AIM2 | V(mU)#(fA)#(mA)(fA)(fA)(fC)(mU)(fU)(mU)(fU)(mA) |
| | targeting) | (fC)(mA)(fA)#(mA)#(fG)#(mA)#(mA)#(mG)#(mA)#(fA) |
| 12 | D-NTC-sense (AIM2 | (C12)(SB)(C6)(SB)(dT)(dT)(Cy3)#(mA)#(mU)#(mU)(fG) |
| | experiments) | (mA)(fC)(mA)(fA)(mA)(fU)(mA)(mC)(mG)(fA)#(mU)#(mA) |
| 13 | NTC-antisense (AIM2 | V(mU)#(fA)#(mU)(fC)(fG)(fU)(mA)(fU)(mU)(fU)(mG) |
| | experiments) | (fU)(mC)(fA)#(mA)#(fU)#(mC)#(mU)#(mU)#(fU)#(mU) |
| 14 | D-sense (tumor gMFI | Same as 9 |
| | experiments) | |
| 15 | DCA-sense (tumor | Cy3(mC)#(mU)#(mU)(fU)(mG)(fU)(mA)(fA)(mA)(fA) |
| | gMFI experiments) | (mG)(mU)(mU)(fU)#(mU)#(mA)(dT)(dT)-DCAv1 |
| 16 | Unc-sense (tumor | Cy3#(mC)#(mU)#(mU)(fU)(mG)(fU)(mA)(fA)(mA)(fA) |
| | gMFI experiments) | (mG)(mU)(mU)(fU)#(mU)#(mA)(dT)(dT) |
| 17 | Antisense (tumor gMFI | V(mU)#(fA)#(mA)(fA)(fA)(fC)(mU)(fU)(mU)(fU)(mA) |
| | experiments) | (fC)(mA)(fA)#(mA)#(fG)#(mA)#(mA)#(mG)#(mA)#(fA) |

V is vinyl phosphonate,
m = 2′omethyl,
f = 2′fluoro,
# = phosphorothioate backbone
d = DNA base

### Example 2. Injection Of Lipid-Conjugated siRNAs Into Mice

Animal experiments of siRNA conjugates were performed in accordance with animal care ethics approval and guidelines of University of Massachusetts Medical School Institutional Animal Care and Use Committee (IACUC; Protocol #202000010) at the RNA therapeutics institute. In all experiments, 7 to 8-week-old female FVB/NJ mice (The Jackson Laboratory) were used and were injected s.c. or i.v. with either non-targeting control siRNA (NTC), or lipid-conjugated siRNA (n=5-6 per group) at a concentration of 20 mg/kg unless specified.

For distribution studies, 3 mice per sample were injected. For efficacy studies, 5-8 to mice per sample, per gene were studied. For toxicity studies, 3 mice per sample were injected.

### Example 3. In Vivo mRNA Silencing Experiments

At 1-week post-injection, mice were euthanized and perfused with PBS. Tissues were collected and stored in RNAlater (Sigma) at 4° C. overnight. mRNA was quantified using the QuantiGene 2.0 Assay (Affymetrix). The 1.5-mm punches (three punches per tissue) were placed in QIAGEN Collection Microtubes holding 3-mm tungsten beads and lysed in 300 μl Homogenizing Buffer (Affymetrix) containing 0.2 mg/ml proteinase K (Invitrogen) using a QIAGEN TissueLyser II. Samples were then centrifuged at 1000×g for 10 min and incubated for 1 h at 55° C. to 60° C. Lysates and diluted probe sets (mouse Htt, mouse Ppib or mouse Hprt) were added to the bDNA capture plate and signal was amplified and detected. Luminescence was detected on a Tecan M1000 (Tecan, Morrisville, NC, USA).

### Example 4. Peptide Nucleic Acid (PNA) Hybridization Assay

Tissue concentrations of antisense strands were determined using a peptide nucleic acid (PNA) hybridization assay. Tissues punches were placed in QIAGEN Collection Microtubes holding 3-mm tungsten beads and lysed in 300 μl MasterPure tissue lysis solution (EpiCentre) containing 0.2 mg/ml proteinase K (Invitrogen) using a QIAGEN TissueLyser II. Lysates were then centrifuged at 1000×g for 10 min and incubated for 1 h at 55° to 60° C. Sodium dodecyl sulphate (SDS) was precipitated from lysates by adding 20 μl 3 M potassium chloride and pelleted centrifugation at 5000×g for 15 min. Conjugated siRNAs in cleared supernatant were hybridized to a Cy3-labeled PNA probe fully complementary to the antisense strand (PNABio, Thousand Oaks, CA, USA). Samples were analyzed by HPLC (Agilent, Santa Clara, CA, USA) over a DNAPac PA100 anion-exchange column (Thermo Fisher Scientific), in a gradient of sodium perchlorate, as follows: Buffer A: 50% water; 50% acetonitrile; 25 mM Tris-HCl, pH 8.5; 1 mM ethylenediaminetetraacetate. Buffer B: 800 mM sodium perchlorate in buffer A. Gradient conditions: 10% buffer B within 4 min, 50% buffer B for 1 min and 50% to 100% buffer B within 5 min. Cy3 fluorescence was monitored and peaks integrated. Final concentrations were ascertained using calibration curves generated by spiking known quantities of lipid-conjugated siRNA into tissue lysates from an untreated animal. Spiked samples for calibration and experimental samples were processed and analyzed under the same laboratory conditions.

### Example 5. Fluorescence Microscopy

Organs were collected from euthanized mice, washed with PBS, molded with OCT embedding medium and frozen. Frozen sections were cut using Leica Cryostat CM1950 at 5 μm thickness, and slides are left to air dry overnight. Then, the slides are hydrated with PBS buffer for 5 mins twice at room temperature, followed by mounting the coverslip on the slides using ProLong™ Gold Antifade Mountant with DAPI staining for nuclei.

### Example 6. Lipoprotein Size Exclusion Chromatography

For lipoprotein profiling, the same protocol previously described by Osborne was followed (see Osborn, M. F. et al. “Hydrophobicity drives the systemic distribution of lipid-conjugated siRNAs via lipid transport pathways,” Nucleic Acids Research 47, 1070-1081 (2018), incorporated herein by reference in their entirety). Briefly, mice were injected intravenously with 10 mg/kg of Cy3-labeled oligonucleotides. After 15 minutes, whole mouse blood (˜500 μl) was collected in a sterile EDTA-coated tube following cheek incision with a lancet. Samples were spun at 10,000 RPM for 10 minutes at 4° C. 50 μl of plasma was directly injected on Superose 6 Increase 10/300 size exclusion column (GE Healthcare). Oligonucleotide migration was monitored at 570 nm, and lipoprotein protein content was monitored by absorbance at 280 nm. For subcutaneous injections, collection was performed at 1 hour post injection.

### Example 7. Reverse Phase HPLC Analysis Of D-siRNA vs DCA-siRNA

The LC data of oligonucleotides was performed on an Agilent 6530 accurate mass Q-TOF using the following conditions: buffer A: 100 mM 1,1,1,3,3,3-hexafluoroisopropanol (HFIP) and 9 mM triethylamine (TEA) in LC-MS grade water; buffer B:100 mM HFIP and 9 mM TEA in LC-MS grade methanol; column, Agilent AdvanceBio oligonucleotides C18; 5-100% B 11 min; temperature, 60° C.; flow rate, 0.5 ml/min. LC peaks were monitored at 260 nm.

### Example 8. Cell Culture and In Vitro Dose Response

HeLa cells were maintained in DMEM (Cellgro, 10-013-CV) supplemented with 10% fetal bovine serum (FBS) (Gibco, 26140) and 100 U/mL pen/strep (Invitrogen, 15140) and expanded at 37° C. and 5% CO₂. Seven-point dose-response curves were generated by treating HeLa cells with various concentrations of siRNA formulated with RNAiMax (Invitrogen, 13778-150) for 72 h at 37° C. and 5% CO₂. Transfection was carried out in 50:50 DMEM/OptiMEM (Gibco, 31985-070) and 3% FBS with no antibiotics. Cells were lysed by 30-min incubation at 55° C. with diluted QuantiGene (QG) lysis mixture (Invitrogen, QP0524) containing proteinase K (Invitrogen, 25530-049). Gene silencing was assessed by QG branched DNA (bDNA) assay as per manufacturer's instructions (see brief description below) using the following probe sets: mouse Htt (SB-14150) and mouse Hprt (SB-15463). Htt data was normalized to housekeeping Hprt and represented as the percentage of untreated control. n=3.

### Example 9. In Vivo Dose Response

Eight-week-old female mice were injected subcutaneously with a panel of Cy3-labeled siRNA variants. After 24 h, mouse femur bone was dissected, and femur bone marrow cells were isolated by flushing dissected femur bones with PBS using a 23-G needle onto a 70-μm cell strainer placed in a 50-mL conical tube. The bone marrow and spleen were then smashed using a 5-mL plunger, followed by rinsing of the strainer with DMEM media. The cell solution collected in the 50-mL tube was centrifuged at 350 g for 5 min at room temperature and washed once with PBS. Blood cells were lysed using ACK lysis buffer (155 mM NH₄Cl, 12 mM NaHCO₃, 0.1 mM EDTA in DW), followed by washing with DMEM (500× g, 10 min, 4° C.). Next, the cell pellet was suspended with a flow cytometry buffer (0.5% BSA, 2 mM EDTA in DMEM) for flow cytometry analysis.

Leukocyte staining of the cell was with VioGreen-conjugated CD45 antibody (clone REA737, Miltenyi Biotec), APC-conjugated GR-1 antibody (clone REA810, Miltenyi Biotec), FITC-conjugated CD11b antibody (clone REA592, Miltenyi Biotec), PE-Vio770-conjugated CD11c antibody (clone REA754, Miltenyi Biotec), and PE_Vio615 F40/80 (clone REA126, Miltenyi Biotec) for lymphocyte staining, PE-vio770 conjugated CD19 (clone REA749) and APC conjugated CD3 antibody (clone REA641) were used. Cells were stained for 30 min at 4° C. Then, cells were washed twice with 700 μl of flow cytometry buffer and resuspended in flow cytometry buffer containing 1 μM SYTOX™ Blue (Thermo Fisher Scientific). Stained cells were analyzed using a MACSQuant® VYB Flow Cytometer (Miltenyi Biotec) and data were analyzed using FlowJo software (v10.6, BD Biosciences, Ashland, OR, USA).

### Example 10. D-siRNA's Selective Protein Binding Profile to Albumin

A lipoprotein binding profile via size exclusion chromatography, as previously developed by Osborne, was assessed (see Osborn, M. F. et al. “Hydrophobicity drives the systemic distribution of lipid-conjugated siRNAs via lipid transport pathways,” Nucleic Acids Research 47, 1070-1081 (2018), incorporated herein in their entirety).

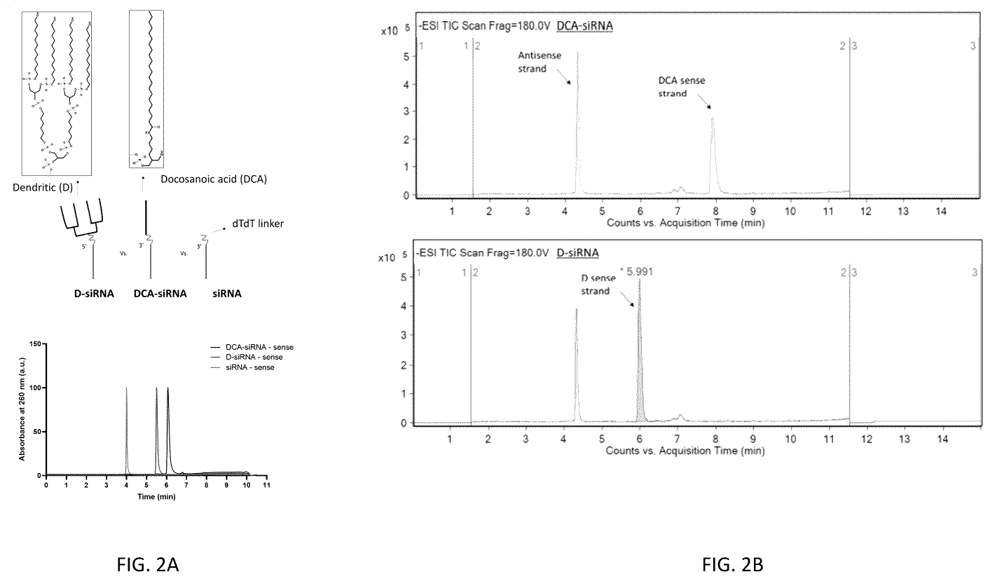

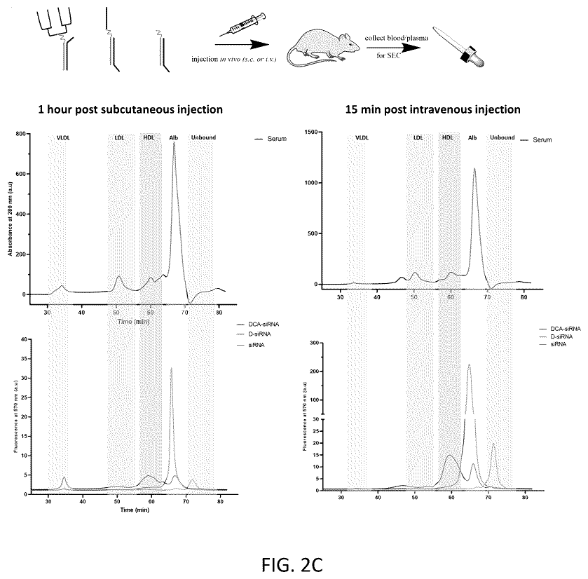

The binding profile of a conjugate partly depends on its hydrophobicity, where more hydrophobic conjugates (such as docosanoic acid, DCA) bind to low-density and high-density lipoproteins (LDL and HDL) in plasma, and to a lesser extent with albumin. The dendritic moiety is larger and has more aliphatic material (69 carbons) compared to DCA (28 carbons). The multiple phosphates that punctuate the structure of the conjugate, in addition to the terminal hydroxyl group, can lead to an increased solubility and prevention of aggregation. Consequently D-siRNA is less hydrophobic than DCA-siRNA, which is evident from lower retention when analyzed by reverse-phase high-performance liquid chromatography (RP-HPLC) (FIG. 2A and FIG. 2B).

Following the hydrophobicity comparison by HPLC, the serum protein binding profile of each conjugate post-injection in mice was examine as previously described. Briefly, animals were injected either subcutaneously (s.c.) or intravenously (i.v.) with Cy3-labeled D-siRNA or DCA-siRNA at a dose of 10 mg/kg, and plasma was collected 15 mins (for i.v.) and 60 minutes (for s.c.) post-injection. The timepoint were chosen to maximize the compounds circulating levels in the blood.

The plasma was then fractionated by size exclusion chromatography, and the Cy-3-siRNA elution time was monitored (570 nm). The Sucrose 6 elusion profile for multiple major plasma proteins (HDL, VLDL, LDL, albumin/globulins) was pre-established. D-siRNA following injection into mice has a retention time of 67 mins, which overlaps with that of albumin, compared to DCA-siRNA which has a multiprotein binding profile associated mostly with LDL and HDL (Error! Reference source not found.B). The protein binding trend was consistent in both routes of administration. This data confirms the selective and high-affinity binding to albumin that is hypothesized to dictate its behavior in vivo as it did in vitro. The data also suggests that D-siRNAs can bind tightly to albumin and circulate as a protein-RNA complex in the blood until it extravasate into various organs and tissues. This is regardless of the mode of injection (subcutaneously or intravenously).

### Example 11. Tumor Delivery of D-siRNA and DCA-siRNA

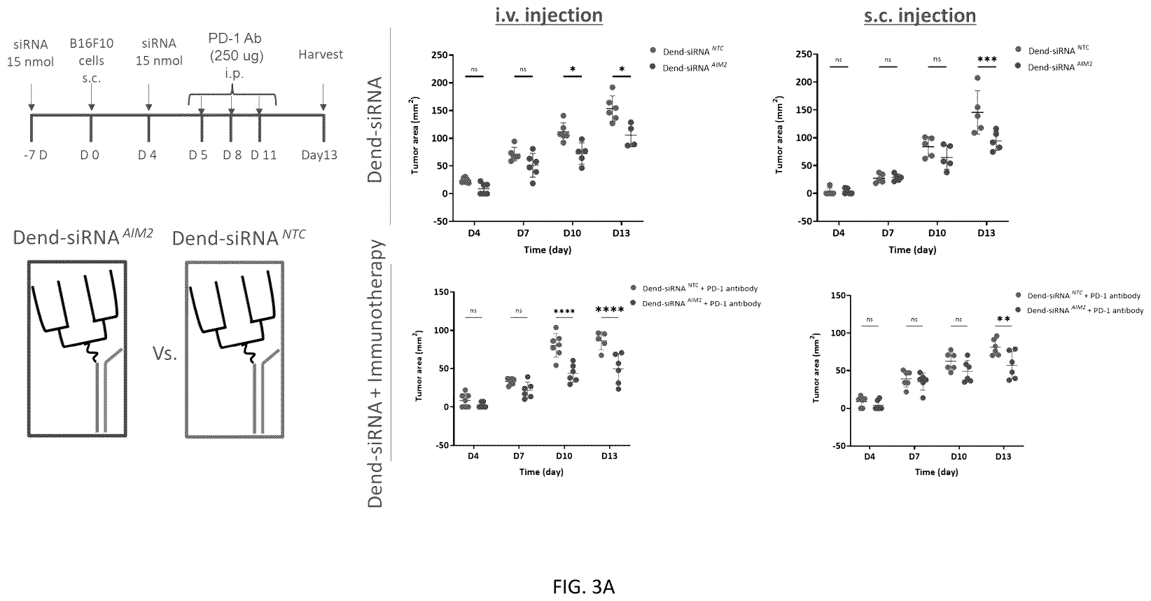

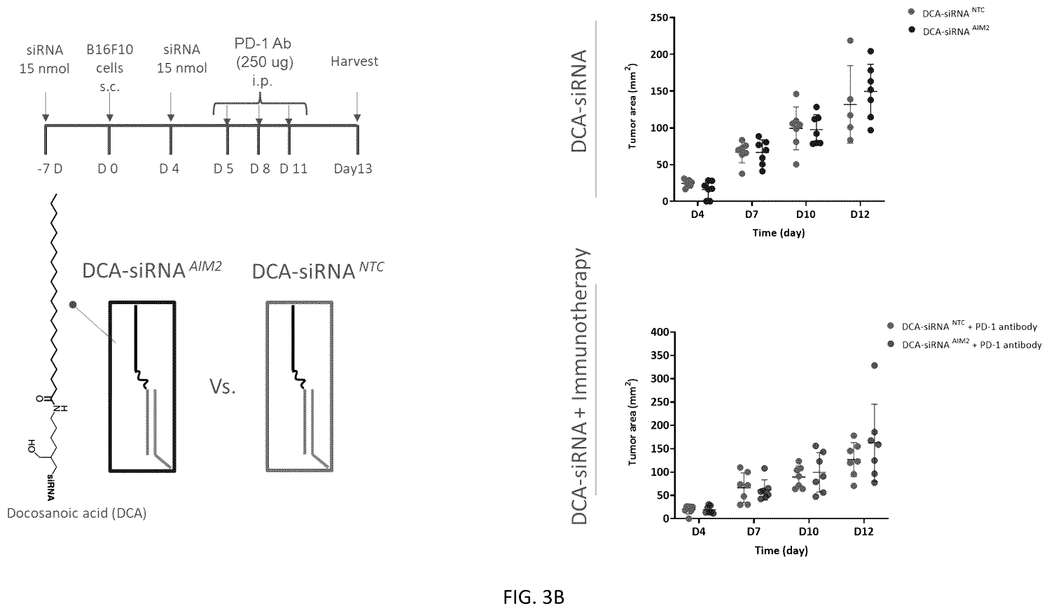

The effect of D-siRNA and DCA-siRNA on tumor growth was assessed (FIG. 3):

Wild type Black 6 mice (males-age 8-12 weeks, n=4-6) were injected subcutaneously (back of mouse) or intravenously (tail vein) with siRNA at day −7 (1 week before tumor inoculation) at a dose of 15 nmol/mouse. Then, B16F10 melanoma cells were injected subcutaneously (right flank) at a n amount of 1 million cells/mouse, and this is designated as day 0. At day 7, another siRNA dose was administered, and mice were euthanized at day 13. Tumor growth was measured using a caliper on days 4, 7, 10, 13. PD-1 was administered on days 5, 8, 11 at a dose of 250 μg/injection, via intraperitoneal injection (abdomen).

It was observed that tumor growth rate was slower in D-siRNA^{AIM2}+anti PD-1 antibody injected group and the D-siRNA AIM2 alone group than in D-siRNA^{NTC}+anti PD-1 antibody injected group or the D-siRNA NTC alone group. The result was observed in both administration routes (i.v., and SC). The DCA-siRNA conjugate was not effective in this experiment. Thus, the data shows that dendrimer-conjugated oligonucleotides, such as siRNA, are effectively delivered to tumor cells and are capable of silencing a target of interest (e.g., AIM2).

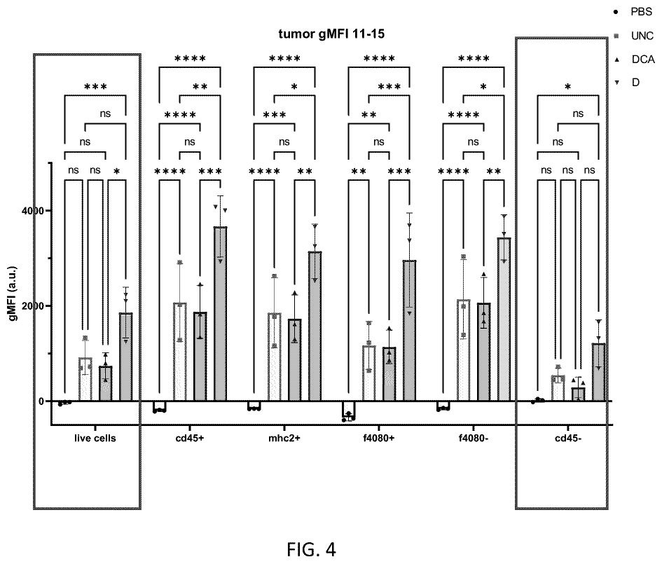

The delivery of D-siRNA and DCA-siRNA to tumor cells was assessed via cy3 signal (FIG. 4):

Tumors were grown for 1 week after mice were injected with 1 million cells of B16F10 cells in the right flank. Then, mice were injected with siRNAs labeled with Cy3 subcutaneously with a 30 nmol amount (around 20 mg/kg). following 24 hours, mice were taken down, organs (tumor, spleen, liver) were collected and processed to obtain single cell suspension. Then these cells were labeled with antibodies to distinguish cell type present (CD45+ for immune cells, MHCII+ presenting immune cells, F40/80+ macrophage cells and f40/80− cells which include dendritic cells). Sample were ran on flow cytometry machine. The results show that dendrimer-conjugated oligonucleotides, such as siRNA, are effectively delivered to tumor cells, with greater delivery compared to a DCA conjugated siRNA.

### Example 12. Safety Profile of D-siRNA

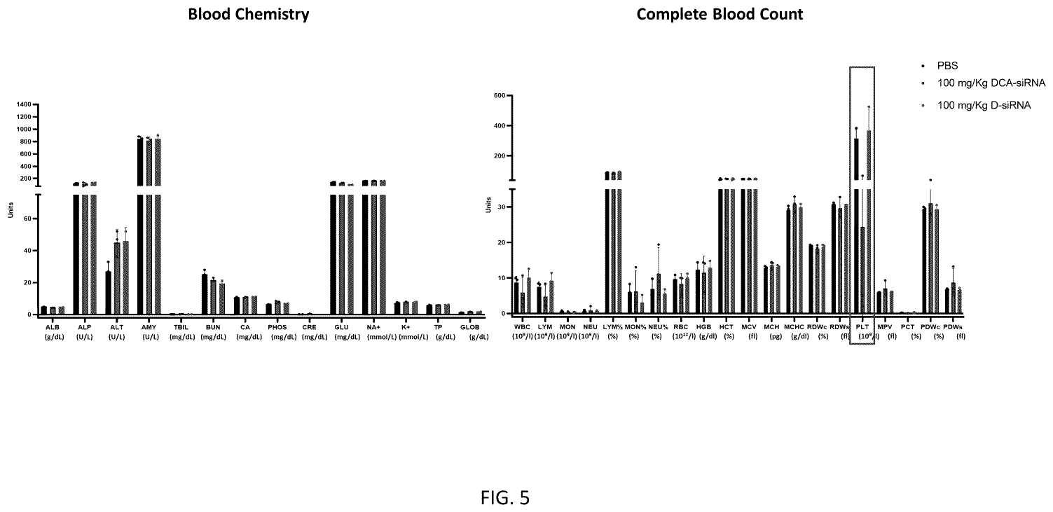

A toxicity test on D-siRNA was performed, where an escalated dose of 100 mg/Kg were injected in mice and blood chemistry and complete blood counts (CBC) at 24 h were monitored (FIG. 5). The data showed no significant alterations to any parameter measured when mice were injected with D-siRNA. However, a clear platelets level drop was observed with DCA-siRNA. This is associated with increased risk of stroke and is an undesirable outcome.

### Example 13. Activity Comparison Between 5′ and 3′ Conjugated D-siRNA

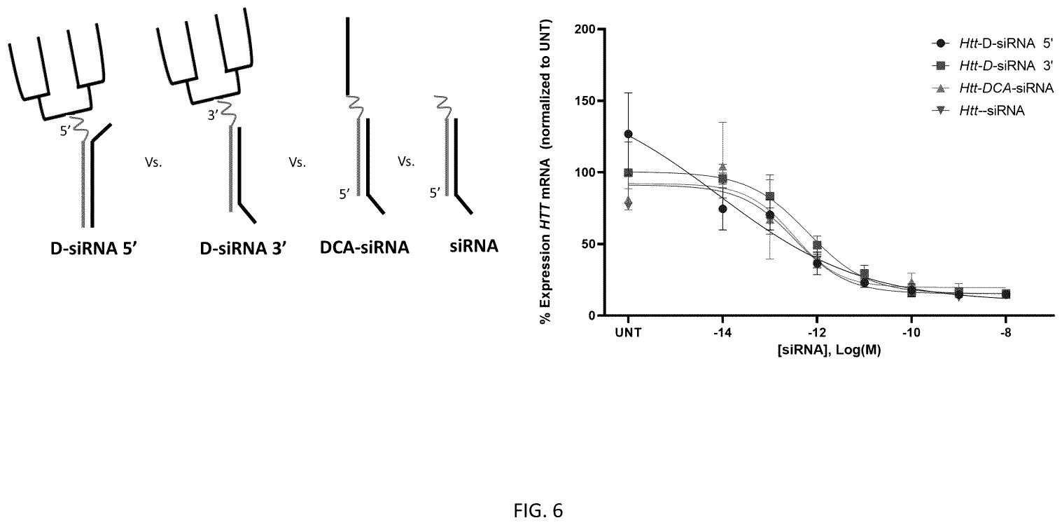

The impact on the directionality of the dendritic conjugate on the D-siRNA activity was assessed (FIG. 6). A 3′ conjugated D-siRNA was synthesized by using reverse amidites to build the sense strand. Htt-targeting D-siRNAs, along with DCA-siRNA and unconjugated-siRNA were transfected into HeLa cells in a dose dependent manner (n=3) for 3 days, and then mRNA silencing efficacy was quantified using QuantiGene® (Affymetrix), normalized to a housekeeping gene, Hprt (Hypoxanthine-guanine phosphoribosyl transferase) and presented as percent untreated control. No significant difference was observed, confirming that D-siRNA is equally function if placed on 5′ or 3′ end of sense strand.

### INCORPORATION BY REFERENCE

The contents of all cited references (including literature references, patents, patent applications, patent publications, and websites) that maybe cited throughout this application are hereby expressly incorporated by reference in their entirety for any purpose, as are the references cited therein. The disclosure will employ, unless otherwise indicated, conventional techniques of immunology, molecular biology and cell biology, which are well known in the art.

The present disclosure also incorporates by reference in their entirety techniques well known in the field of molecular biology and drug delivery. These techniques include, but are not limited to, techniques described in the following publications:
Nguyen, V. H. & Lee, B.-J. Protein corona: a new approach for nanomedicine design. Int J Nanomedicine 12, 3137-3151 (2017).
Banker, M. J. & Clark, T. H. Plasma/serum protein binding determinations. Curr Drug Metab 9, 854-859 (2008).
Zhao, Z., Ukidve, A., Krishnan, V. & Mitragotri, S. Effect of physicochemical and surface properties on in vivo fate of drug nanocarriers. Advanced Drug Delivery Reviews 143, 3-21 (2019).
Albanese, A., Tang, P. S. & Chan, W. C. W. The Effect of Nanoparticle Size, Shape, and Surface Chemistry on Biological Systems. Annual Review of Biomedical Engineering 14, 1-16 (2012).
de Castro, C. E. et al. The Protein Corona Conundrum: Exploring the Advantages and Drawbacks of its Presence around Amphiphilic Nanoparticles. Bioconjugate Chemistry 31, 2638-2647 (2020).
Eckstein, F. Phosphorothioates, essential components of therapeutic oligonucleotides. Nucleic Acid Ther 24, 374-387 (2014).
Agarwal, S. et al. Impact of Serum Proteins on the Uptake and RNAi Activity of GalNAc-Conjugated siRNAs. Nucleic Acid Therapeutics 31, 309-315 (2021).
Francia, V., Schiffelers, R. M., Cullis, P. R. & Witzigmann, D. The Biomolecular Corona of Lipid Nanoparticles for Gene Therapy. Bioconjugate Chemistry 31, 2046-2059
Osborn, M. F. et al. Hydrophobicity drives the systemic distribution of lipid-conjugated siRNAs via lipid transport pathways. Nucleic Acids Res 47, 1070-1081 (2019).
Kratz, F. Albumin as a drug carrier: design of prodrugs, drug conjugates and nanoparticles. J Control Release 132, 171-183 (2008).
Hoogenboezem, E. N. & Duvall, C. L. Harnessing albumin as a carrier for cancer therapies. Advanced Drug Delivery Reviews 130, 73-89 (2018).
Andersen, J. T. & Sandlie, I. The versatile MHC class I-related FcRn protects IgG and albumin from degradation: implications for development of new diagnostics and therapeutics. Drug metabolism and pharmacokinetics 24, 318-332 (2009).
Schnitzer, J. E. gp60 is an albumin-binding glycoprotein expressed by continuous endothelium involved in albumin transcytosis. American Journal of Physiology—Heart and Circulatory Physiology 262, H246-H254 (1992).
Merlot, A. M., Kalinowski, D. S. & Richardson, D. R. Unraveling the mysteries of serum albumin-more than just a serum protein. Front Physiol 5, 299-299 (2014).
Sarett, S. M. et al. Lipophilic siRNA targets albumin in situ and promotes bioavailability, tumor penetration, and carrier-free gene silencing. Proceedings of the National Academy of Sciences 114, E6490-E6497 (2017).
Liu, H. et al. Structure-based programming of lymph-node targeting in molecular vaccines. Nature 507, 519-522 (2014).
Lau, S. et al. Enhanced Extravasation, Stability and in Vivo Cardiac Gene Silencing via in Situ siRNA—Albumin Conjugation. Molecular Pharmaceutics 9, 71-80 (2012).
Lacroix, A., Edwardson, T. G. W., Hancock, M. A., Dore, M. D. & Sleiman, H. F. Development of DNA Nanostructures for High-Affinity Binding to Human Serum Albumin. Journal of the American Chemical Society 139, 7355-7362 (2017).
Purdie, L., Alexander, C., Spain, S. G. & Magnusson, J. P. Alkyl-Modified Oligonucleotides as Intercalating Vehicles for Doxorubicin Uptake via Albumin Binding. Molecular Pharmaceutics 15, 437-446 (2018).
Bern, M., Sand, K. M. K., Nilsen, J., Sandlie, I. & Andersen, J. T. The role of albumin receptors in regulation of albumin homeostasis: Implications for drug delivery. Journal of Controlled Release 211, 144-162 (2015).
Felber, A. E. et al. The interactions of amphiphilic antisense oligonucleotides with serum proteins and their effects on in vitro silencing activity. Biomaterials 33, 5955-5965 (2012).
Monopoli, M. P., Åberg, C., Salvati, A. & Dawson, K. A. Biomolecular coronas provide the biological identity of nanosized materials. Nature Nanotechnology 7, 779-786 (2012).
Lacroix, A., Fakih, H. H. & Sleiman, H. F. Detailed cellular assessment of albumin-bound oligonucleotides: Increased stability and lower non-specific cell uptake. Journal of Controlled Release 324, 34-46 (2020).
Biscans, A. et al. Docosanoic acid conjugation to siRNA enables functional and safe delivery to skeletal and cardiac muscles. Molecular Therapy 29 (2020).
Biscans, A. et al. Docosanoic acid conjugation to siRNA enables functional and safe delivery to skeletal and cardiac muscles. Molecular Therapy 29, 1382-1394 (2021).
Osborn, M. F. et al. Hydrophobicity drives the systemic distribution of lipid-conjugated siRNAs via lipid transport pathways. Nucleic Acids Research 47, 1070-1081 (2018).
Biscans, A. et al. Diverse lipid conjugates for functional extra-hepatic siRNA delivery in vivo. Nucleic Acids Research 47, 1082-1096 (2018).
Coles, A. H. et al. A High-Throughput Method for Direct Detection of Therapeutic Oligonucleotide-Induced Gene Silencing In Vivo. Nucleic Acid Therapeutics 26, 86-92 (2016).
Godinho, B. M. D. C. et al. Pharmacokinetic Profiling of Conjugated Therapeutic Oligonucleotides: A High-Throughput Method Based Upon Serial Blood Microsampling Coupled to Peptide Nucleic Acid Hybridization Assay. Nucleic Acid Therapeutics 27 (2017).
Godinho, B. et al. Pharmacokinetic Profiling of Conjugated Therapeutic Oligonucleotides: A High-Throughput Method Based Upon Serial Blood Microsampling Coupled to Peptide Nucleic Acid Hybridization Assay. Nucleic Acid Ther 27, 323-334 (2017).
Sand, K. M. K. et al. Unraveling the Interaction between FcRn and Albumin: Opportunities for Design of Albumin-Based Therapeutics. Frontiers in Immunology 5 (2015).
Lomis, N. et al. Albumin Nanoparticle Formulation for Heart-Targeted Drug Delivery: In Vivo Assessment of Congestive Heart Failure. Pharmaceuticals (Basel) 14 (2021).
John, T. A. et al. Evidence for the role of alveolar epithelial gp60 in active transalveolar albumin transport in the rat lung. The Journal of Physiology 533, 547-559 (2001).
Woods, A. et al. In vivo biocompatibility, clearance, and biodistribution of albumin vehicles for pulmonary drug delivery. Journal of controlled release: official journal of the Controlled Release Society 210, 1-9 (2015).
Dheilly, E. et al. Selective Blockade of the Ubiquitous Checkpoint Receptor CD47 Is Enabled by Dual-Targeting Bispecific Antibodies. Mol Ther 25, 523-533 (2017).
Shin, M. et al. Intratracheally administered LNA gapmer antisense oligonucleotides induce robust gene silencing in mouse lung fibroblasts. Nucleic Acids Res 50, 8418-8430 (2022).
Didiot, M. C. et al. Nuclear Localization of Huntingtin mRNA Is Specific to Cells of Neuronal Origin. Cell Rep 24, 2553-2560.e2555 (2018).
Biscans, A. et al. Diverse lipid conjugates for functional extra-hepatic siRNA delivery in vivo. Nucleic acids research 47, 1082-1096 (2019).
Nikan, M. et al. Docosahexaenoic acid conjugation enhances distribution and safety of siRNA upon local administration in mouse brain. Molecular Therapy-Nucleic Acids 5, e344 (2016).
Alterman, J. F. et al. Hydrophobically modified siRNAs silence huntingtin mRNA in primary neurons and mouse brain. Molecular Therapy-Nucleic Acids 4, e266 (2015).
Davis, S. M. et al. Chemical optimization of siRNA for safe and efficient silencing of placental sFLT1. Mol Ther Nucleic Acids 29, 135-149 (2022).
Ly, S., Echeverria, D., Sousa, J. & Khvorova, A. Single-Stranded Phosphorothioated Regions Enhance Cellular Uptake of Cholesterol-Conjugated siRNA but Not Silencing Efficacy. Molecular Therapy—Nucleic Acids 21, 991-1005 (2020).
Malhotra, A. & Mittal, B. R. SiRNA gene therapy using albumin as a carrier. Pharmacogenetics and Genomics 24 (2014).
Hanif, F., Muzaffar, K., Perveen, K., Malhi, S. M. and Simjee Sh, U. (2017) Glioblastoma Multiforme: A Review of its Epidemiology and Pathogenesis through Clinical Presentation and Treatment. Asian Pac J Cancer Prev, 18, 3-9.
Wu, W., Klockow, J. L., Zhang, M., Lafortune, F., Chang, E., Jin, L., Wu, Y. and Daldrup-Link, H. E. (2021) Glioblastoma multiforme (GBM): An overview of current therapies and mechanisms of resistance. Pharmacol Res, 171, 105780.
Patel, A. P., Tirosh, I., Trombetta, J. J., Shalek, A. K., Gillespie, S. M., Wakimoto, H., Cahill, D. P., Nahed, B. V., Curry, W. T., Martuza, R. L. et al. (2014) Single-cell RNA-seq highlights intratumoral heterogeneity in primary glioblastoma. Science, 344, 1396-1401.
Neftel, C., Laffy, J., Filbin, M. G., Hara, T., Shore, M. E., Rahme, G. J., Richman, A. R., Silverbush, D., Shaw, M. L., Hebert, C. M. et al. (2019) An Integrative Model of Cellular States, Plasticity, and Genetics for Glioblastoma. Cell, 178, 835-849 e821.
Khvorova, A. and Watts, J. K. (2017) The chemical evolution of oligonucleotide therapies of clinical utility. Nat Biotechnol, 35, 238-248.
Bogdahn, U., Hau, P., Stockhammer, G., Venkataramana, N. K., Mahapatra, A. K., Suri, A., Balasubramaniam, A., Nair, S., Oliushine, V., Parfenov, V. et al. (2011) Targeted therapy for high-grade glioma with the TGF-beta2 inhibitor trabedersen: results of a randomized and controlled phase IIb study. Neuro Oncol, 13, 132-142.
Grossman, S. A., Alavi, J. B., Supko, J. G., Carson, K. A., Priet, R., Dorr, F. A., Grundy, J. S. and Holmlund, J. T. (2005) Efficacy and toxicity of the antisense oligonucleotide aprinocarsen directed against protein kinase C-alpha delivered as a 21-day continuous intravenous infusion in patients with recurrent high-grade astrocytomas. Neuro Oncol, 7, 32-40.
Salloum, R., Hummel, T. R., Kumar, S. S., Dorris, K., Li, S., Lin, T., Daryani, V. M., Stewart, C. F., Miles, L., Poussaint, T. Y. et al. (2016) A molecular biology and phase II study of imetelstat (GRN163L) in children with recurrent or refractory central nervous system malignancies: a pediatric brain tumor consortium study. J Neurooncol, 129, 443-451.
Bennett, C. F., Kordasiewicz, H. B. and Cleveland, D. W. (2021) Antisense Drugs Make Sense for Neurological Diseases. Annu Rev Pharmacol Toxicol, 61, 831-852.
Alterman, J. F., Godinho, B., Hassler, M. R., Ferguson, C. M., Echeverria, D., Sapp, E., Haraszti, R. A., Coles, A. H., Conroy, F., Miller, R. et al. (2019) A divalent siRNA chemical scaffold for potent and sustained modulation of gene expression throughout the central nervous system. Nat Biotechnol, 37, 884-894.
GuhaSarkar, D., Su, Q., Gao, G. and Sena-Esteves, M. (2016) Systemic AAV9-IFNbeta gene delivery treats highly invasive glioblastoma. Neuro Oncol, 18, 1508-1518.
Tchoghandjian, A., Baeza, N., Colin, C., Cayre, M., Metellus, P., Beclin, C., Ouafik, L. and Figarella-Branger, D. (2010) A2B5 cells from human glioblastoma have cancer stem cell properties. Brain Pathol, 20, 211-221.
Valente, V., Teixeira, S. A., Neder, L., Okamoto, O. K., Oba-Shinjo, S. M., Marie, S. K., Scrideli, C. A., Paco-Larson, M. L. and Carlotti, C. G., Jr. (2009) Selection of suitable housekeeping genes for expression analysis in glioblastoma using quantitative RT-PCR. BMC Mol Biol, 10, 17.
Wakimoto, H., Kesari, S., Farrell, C. J., Curry, W. T., Jr., Zaupa, C., Aghi, M., Kuroda, T., Stemmer-Rachamimov, A., Shah, K., Liu, T. C. et al. (2009) Human glioblastoma-derived cancer stem cells: establishment of invasive glioma models and treatment with oncolytic herpes simplex virus vectors. Cancer Res, 69, 3472-3481.
Osborn, M. F., Coles, A. H., Golebiowski, D., Echeverria, D., Moazami, M. P., Watts, J. K., Sena-Esteves, M. and Khvorova, A. (2018) Efficient Gene Silencing in Brain Tumors with Hydrophobically Modified siRNAs. Mol Cancer Ther, 17, 1251-1258.
Teplyuk, N. M., Uhlmann, E. J., Gabriely, G., Volfovsky, N., Wang, Y., Teng, J., Karmali, P., Marcusson, E., Peter, M., Mohan, A. et al. (2016) Therapeutic potential of targeting microRNA-10b in established intracranial glioblastoma: first steps toward the clinic. EMBO Mol Med, 8, 268-287.
Puchalski, R. B., Shah, N., Miller, J., Dalley, R., Nomura, S. R., Yoon, J. G., Smith, K. A., Lankerovich, M., Bertagnolli, D., Bickley, K. et al. (2018) An anatomic transcriptional atlas of human glioblastoma. Science, 360, 660-663.
Mortberg, M. A., Gentile, J. E., Nadaf, N. M., Vanderburg, C., Simmons, S., Dubinsky, D., Slamin, A., Maldonado, S., Petersen, C. L., Jones, N. et al. (2023) A single-cell map of antisense oligonucleotide activity in the brain. Nucleic Acids Res, 51, 7109-7124.
Kordasiewicz, H. B., Stanek, L. M., Wancewicz, E. V., Mazur, C., McAlonis, M. M., Pytel, K. A., Artates, J. W., Weiss, A., Cheng, S. H., Shihabuddin, L. S. et al. (2012) Sustained therapeutic reversal of Huntington's disease by transient repression of huntingtin synthesis. Neuron, 74, 1031-1044.
Khvorova, A., Ferguson, C. and Davis, S. (2023). Oligonucleotides for App Modulation. PCT Patent Application WO/2023173061A2, filed Mar. 10, 2023
Lucero, R., Zappulli, V., Sammarco, A., Murillo, O. D., Cheah, P. S., Srinivasan, S., Tai, E., Ting, D. T., Wei, Z., Roth, M. E. et al. (2020) Glioma-Derived miRNA-Containing Extracellular Vesicles Induce Angiogenesis by Reprogramming Brain Endothelial Cells. Cell Rep, 30, 2065-2074 e2064.
Teng, J., da Hora, C. C., Kantar, R. S., Nakano, I., Wakimoto, H., Batchelor, T. T., Chiocca, E. A., Badr, C. E. and Tannous, B. A. (2017) Dissecting inherent intratumor heterogeneity in patient-derived glioblastoma culture models. Neuro Oncol, 19, 820-832.
Tchoghandjian, A., Baeza-Kallee, N., Beclin, C., Metellus, P., Colin, C., Ducray, F., Adelaide, J., Rougon, G. and Figarella-Branger, D. (2012) Cortical and subventricular zone glioblastoma-derived stem-like cells display different molecular profiles and differential in vitro and in vivo properties. Ann Surg Oncol, 19 Suppl 3, 5608-619.
Souberan, A., Cappai, J., Chocry, M., Nuccio, C., Raujol, J., Colin, C., Lafitte, D., Kovacic, H., Quillien, V., Baeza-Kallee, N. et al. (2019) Inhibitor of Apoptosis Proteins Determines Glioblastoma Stem-Like Cell Fate in an Oxygen-Dependent Manner. Stem Cells, 37, 731-742.
Vickers, T. A., Wyatt, J. R. and Freier, S. M. (2000) Effects of RNA secondary structure on cellular antisense activity. Nucleic Acids Res, 28, 1340-1347.
Gredell, J. A., Berger, A. K. and Walton, S. P. (2008) Impact of target mRNA structure on siRNA silencing efficiency: A large-scale study. Biotechnol Bioeng, 100, 744-755.
Vickers, T. A., Koo, S., Bennett, C. F., Crooke, S. T., Dean, N. M. and Baker, B. F. (2003) Efficient reduction of target RNAs by small interfering RNA and RNase H-dependent antisense agents. A comparative analysis. J Biol Chem, 278, 7108-7118.
Lee, H. N., Jeong, M. S. and Jang, S. B. (2021) Molecular Characteristics of Amyloid Precursor Protein (APP) and Its Effects in Cancer. Int J Mol Sci, 22.
Liu, T., Ren, D., Zhu, X., Yin, Z., Jin, G., Zhao, Z., Robinson, D., Li, X., Wong, K., Cui, K. et al. (2013) Transcriptional signaling pathways inversely regulated in Alzheimer's disease and glioblastoma multiform. Sci Rep, 3, 3467.
Chasseigneaux, S. and Allinquant, B. (2012) Functions of Abeta, sAPPalpha and sAPPbeta: similarities and differences. J Neurochem, 120 Suppl 1, 99-108.
Juliano, R. L., Wang, L., Tavares, F., Brown, E. G., James, L., Ariyarathna, Y., Ming, X., Mao, C. and Suto, M. (2018) Structure-activity relationships and cellular mechanism of action of small molecules that enhance the delivery of oligonucleotides. Nucleic Acids Res, 46, 1601-1613.
Juliano, R. L. (2016) The delivery of therapeutic oligonucleotides. Nucleic Acids Res, 44, 6518-6548.
Juliano, R. L., Ming, X. and Nakagawa, O. (2012) The chemistry and biology of oligonucleotide conjugates. Acc Chem Res, 45, 1067-1076.
Wolfrum, C., Shi, S., Jayaprakash, K. N., Jayaraman, M., Wang, G., Pandey, R. K., Rajeev, K. G., Nakayama, T., Charrise, K., Ndungo, E. M. et al. (2007) Mechanisms and optimization of in vivo delivery of lipophilic siRNAs. Nat Biotechnol, 25, 1149-1157.
Ostergaard, M. E., Jackson, M., Low, A., A, E. C., R, G. L., Peralta, R. Q., Yu, J., Kinberger, G. A., Dan, A., Carty, R. et al. (2019) Conjugation of hydrophobic moieties enhances potency of antisense oligonucleotides in the muscle of rodents and non-human primates. Nucleic Acids Res, 47, 6045-6058.
Brown, K. M., Nair, J. K., Janas, M. M., Anglero-Rodriguez, Y. I., Dang, L. T. H., Peng, H., Theile, C. S., Castellanos-Rizaldos, E., Brown, C., Foster, D. et al. (2022) Expanding RNAi therapeutics to extrahepatic tissues with lipophilic conjugates. Nat Biotechnol, 40, 1500-1508.
Wang, S., Allen, N., Liang, X. H. and Crooke, S. T. (2018) Membrane Destabilization Induced by Lipid Species Increases Activity of Phosphorothioate-Antisense Oligonucleotides. Mol Ther Nucleic Acids, 13, 686-698.
Biscans, A., Coles, A., Echeverria, D. and Khvorova, A. (2019) The valency of fatty acid conjugates impacts siRNA pharmacokinetics, distribution, and efficacy in vivo. J Control Release, 302, 116-125.
Tang, Q., Fakih, H. H., Zain Ui Abideen, M., Hildebrand, S. R., Afshari, K., Gross, K. Y., Sousa, J., Maebius, A. S., Bartholdy, C., Sogaard, P. P. et al. (2023) Rational design of a JAK1-selective siRNA inhibitor for the modulation of autoimmunity in the skin. Nat Commun, 14, 7099.
Fakih, H. H., Tang, Q., Summers, A., Shin, M., Buchwald, J. E., Gagnon, R., Hariharan, V. N., Echeverria, D., Cooper, D. A., Watts, J. K. et al. (2023) Dendritic amphiphilic siRNA: Selective albumin binding, in vivo efficacy, and low toxicity. Mol Ther Nucleic Acids, 34, 102080.
Hagedorn, P. H., Brown, J. M., Easton, A., Pierdomenico, M., Jones, K., Olson, R. E., Mercer, S. E., Li, D., Loy, J., Hog, A. M. et al. (2022) Acute Neurotoxicity of Antisense Oligonucleotides After Intracerebroventricular Injection Into Mouse Brain Can Be Predicted from Sequence Features. Nucleic Acid Ther, 32, 151-162.
Jia, C., Lei Mon, S. S., Yang, Y., Katsuyama, M., Yoshida-Tanaka, K., Nagata, T., Yoshioka, K. and Yokota, T. (2023) Change of intracellular calcium level causes acute neurotoxicity by antisense oligonucleotides via CSF route. Mol Ther Nucleic Acids, 31, 182-196.
Tran, H., Moazami, M. P., Yang, H., McKenna-Yasek, D., Douthwright, C. L., Pinto, C., Metterville, J., Shin, M., Sanil, N., Dooley, C. et al. (2022) Suppression of mutant C9orf72 expression by a potent mixed backbone antisense oligonucleotide. Nat Med, 28, 117-124.
Petrecca, K., Guiot, M. C., Panet-Raymond, V. and Souhami, L. (2013) Failure pattern following complete resection plus radiotherapy and temozolomide is at the resection margin in patients with glioblastoma. J Neurooncol, 111, 19-23.
Liu, X., Wang, W., Samarsky, D., Liu, L., Xu, Q., Zhang, W., Zhu, G., Wu, P., Zuo, X., Deng, H. et al. (2014) Tumor-targeted in vivo gene silencing via systemic delivery of cRGD-conjugated siRNA. Nucleic Acids Res, 42, 11805-11817.
Orellana, E. A., Tenneti, S., Rangasamy, L., Lyle, L. T., Low, P. S. and Kasinski, A. L. (2017) FolamiRs: Ligand-targeted, vehicle-free delivery of microRNAs for the treatment of cancer. Sci Transl Med, 9.
Song, E., Zhu, P., Lee, S. K., Chowdhury, D., Kussman, S., Dykxhoorn, D. M., Feng, Y., Palliser, D., Weiner, D. B., Shankar, P. et al. (2005) Antibody mediated in vivo delivery of small interfering RNAs via cell-surface receptors. Nat Biotechnol, 23, 709-717.
He, S., Cen, B., Liao, L., Wang, Z., Qin, Y., Wu, Z., Liao, W., Zhang, Z. and Ji, A. (2017) A tumor-targeting cRGD-EGFR siRNA conjugate and its anti-tumor effect on glioblastoma in vitro and in vivo. Drug Deliv, 24, 471-481.
Abdelaal, A. M. and Kasinski, A. L. (2021) Ligand-mediated delivery of RNAi-based therapeutics for the treatment of oncological diseases. NAR Cancer, 3, zcab030.
Alam, M. R., Ming, X., Fisher, M., Lackey, J. G., Rajeev, K. G., Manoharan, M. and Juliano, R. L. (2011) Multivalent cyclic RGD conjugates for targeted delivery of small interfering RNA. Bioconjug Chem, 22, 1673-1681.
Gilboa-Geffen, A., Hamar, P., Le, M. T., Wheeler, L. A., Trifonova, R., Petrocca, F., Wittrup, A. and Lieberman, J. (2015) Gene Knockdown by EpCAM Aptamer-siRNA Chimeras Suppresses Epithelial Breast Cancers and Their Tumor-Initiating Cells. Mol Cancer Ther, 14, 2279-2291.
Zhang, Y., Xie, X., Yeganeh, P. N., Lee, D. J., Valle-Garcia, D., Meza-Sosa, K. F., Junqueira, C., Su, J., Luo, H. R., Hide, W. et al. (2021) Immunotherapy for breast cancer using EpCAM aptamer tumor-targeted gene knockdown. Proc Natl Acad Sci USA, 118.
Okada, M., Suzuki, S., Togashi, K., Sugai, A., Yamamoto, M. and Kitanaka, C. (2021) Targeting Folate Metabolism Is Selectively Cytotoxic to Glioma Stem Cells and Effectively Cooperates with Differentiation Therapy to Eliminate Tumor-Initiating Cells in Glioma Xenografts. Int J Mol Sci, 22.
Miner, M. W. G., Liljenback, H., Virta, J., Karna, S., Viitanen, R., Elo, P., Gardberg, M., Teuho, J., Saipa, P., Rajander, J. et al. (2023) High folate receptor expression in gliomas can be detected in vivo using folate-based positron emission tomography with high tumor-to-brain uptake ratio divulging potential future targeting possibilities. Front Immunol, 14, 1145473.
Scaranti, M., Cojocaru, E., Banerjee, S. and Banerji, U. (2020) Exploiting the folate receptor alpha in oncology. Nat Rev Clin Oncol, 17, 349-359.
Zhao, R., Matherly, L. H. and Goldman, I. D. (2009) Membrane transporters and folate homeostasis: intestinal absorption and transport into systemic compartments and tissues. Expert Rev Mol Med, 11, e4.
Crooke, S. T., Vickers, T. A. and Liang, X. H. (2020) Phosphorothioate modified oligonucleotide-protein interactions. Nucleic Acids Res, 48, 5235-5253.
Joo, K. M., Kim, J., Jin, J., Kim, M., Seol, H. J., Muradov, J., Yang, H., Choi, Y. L., Park, W. Y., Kong, D. S. et al. (2013) Patient-specific orthotopic glioblastoma xenograft models recapitulate the histopathology and biology of human glioblastomas in situ. Cell Rep, 3, 260-273.
Soifer, H. S., Koch, T., Lai, J., Hansen, B., Hoeg, A., Oerum, H. and Stein, C. A. (2012) Silencing of gene expression by gymnotic delivery of antisense oligonucleotides. Methods Mol Biol, 815, 333-346.
Gonzalez-Barriga, A., Nillessen, B., Kranzen, J., van Kessel, I. D. G., Croes, H. J. E., Aguilera, B., de Visser, P. C., Datson, N. A., Mulders, S. A. M., van Deutekom, J. C. T. et al. (2017) Intracellular Distribution and Nuclear Activity of Antisense Oligonucleotides After Unassisted Uptake in Myoblasts and Differentiated Myotubes In Vitro. Nucleic Acid Ther, 27, 144-158.
Wakimoto, H., Mohapatra, G., Kanai, R., Curry, W. T., Jr., Yip, S., Nitta, M., Patel, A. P., Barnard, Z. R., Stemmer-Rachamimov, A. O., Louis, D. N. et al. (2012) Maintenance of primary tumor phenotype and genotype in glioblastoma stem cells. Neuro Oncol, 14, 132-144.
Lee, J., Kotliarova, S., Kotliarov, Y., Li, A., Su, Q., Donin, N. M., Pastorino, S., Purow, B. W., Christopher, N., Zhang, W. et al. (2006) Tumor stem cells derived from glioblastomas cultured in bFGF and EGF more closely mirror the phenotype and genotype of primary tumors than do serum-cultured cell lines. Cancer Cell, 9, 391-403.
Zhang, Q., Yang, L., Liu, Y. H., Wilkinson, J. E. and Krainer, A. R. (2023) Antisense oligonucleotide therapy for H3.3K27M diffuse midline glioma. Sci Transl Med, 15, eadd8280.

### EQUIVALENTS

The disclosure may be embodied in other specific forms without departing from the spirit or essential characteristics thereof. The foregoing embodiments are therefore to be considered in all respects illustrative rather than limiting of the disclosure. Scope of the disclosure is thus indicated by the appended claims rather than by the foregoing description, and all changes that come within the meaning and range of equivalency of the claims are therefore intended to be embraced herein.

1. A method for delivering an oligonucleotide conjugate to a tumor of a subject, or a method for treating cancer in the subject, or a method for administering a therapeutically effective amount of the oligonucleotide conjugate to the tumor of the subject, the method comprising administering the oligonucleotide conjugate to the subject, wherein the oligonucleotide conjugate comprises:
(a) an oligonucleotide comprising a 5′ end, a 3′ end, and complementarity to a target nucleic acid; and
(b) a dendron that is linked to the oligonucleotide and comprises an end group, a phosphate group, and/or a hydrophobic chain; and
wherein the oligonucleotide conjugate is formulated for tumor delivery.

2. The method of claim 1, wherein the end group is a hydrophilic group comprising a hydroxide, an amine, a phosphate, a sulfur, and/or a sugar; a hydrophobic group comprising an amine, an amide, an ether, an ester, a N- or O-containing heterocycle, a thiol, a thioether, and/or a saturated or unsaturated C₁₋₂₄ alkyl chain; and/or one or more aromatic rings.

3. The method of claim 1, wherein the hydrophobic chain is a saturated or unsaturated C₁₋₂₄ alkyl group.

4. The method of claim 1, wherein the dendron comprises two branches, four branches, or eight branches.

5-7. (canceled)

8. The method of claim 1, wherein the oligonucleotide corresponds to an antisense oligonucleotide or a siRNA.

9. The method of claim 8, wherein the siRNA comprises a sense strand and an antisense strand.

10. The method of claim 9, wherein the dendron is linked to the 5′ end and/or the 3′ end of the sense strand or to the 5′ end and/or the 3′ end of the antisense strand.

11. (canceled)

12. The method of claim 9, wherein the antisense strand comprises about 15 nucleotides to about 25 nucleotides in length, or wherein the antisense strand is 20 nucleotides in length, 21 nucleotides in length, or 22 nucleotides in length.

13. The method of claim 9, wherein the sense strand comprises about 15 nucleotides to about 25 nucleotides in length, or wherein the sense strand is 15 nucleotides in length, 16 nucleotides in length, 18 nucleotides in length, or 20 nucleotides in length.

14-15. (canceled)

16. The method of claim 9, wherein the siRNA comprises a double-stranded region of 15 base pairs to 20 base pairs, or wherein the siRNA comprises a double-stranded region of 15 base pairs, 16 base pairs, 18 base pairs, or 20 base pairs.

17-20. (canceled)

21. The method of claim 9, wherein the siRNA comprises at least one modified nucleotide.

22. (canceled)

23. The method of claim 8, wherein the siRNA comprises at least one modified internucleotide linkage.

24-29. (canceled)

30. The method of claim 9, wherein the antisense strand comprises a 5′-phosphate, a 5′-alkyl phosphonate, a 5′-alkylene phosphonate, or a 5′-alkenyl phosphonate.

31-33. (canceled)

34. The method of claim 1, wherein the oligonucleotide conjugate has a structure of Formula I: 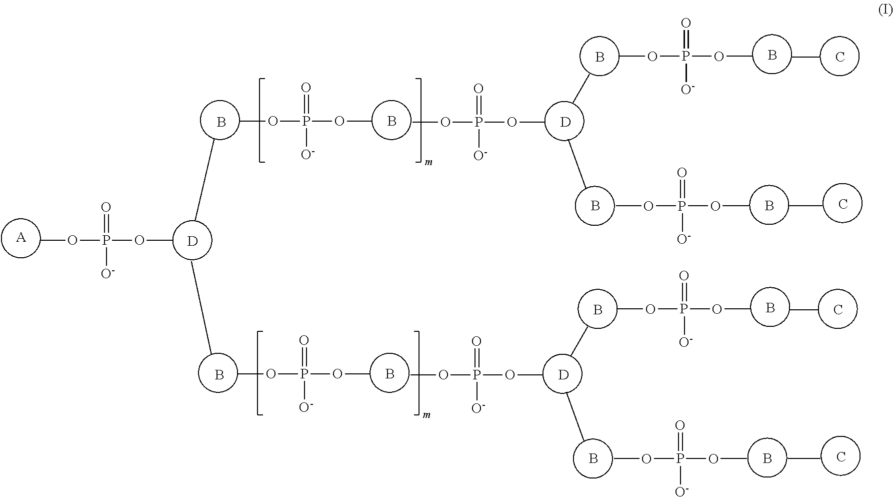
wherein:
A is an oligonucleotide;
B, for each occurrence, independently comprises one or more hydrophobic chains, amines, amides, esters, N- or O-containing heterocycles, thioethers, disulfides, and/or aromatic rings, wherein the one of more hydrophobic chains comprise a saturated or unsaturated C₁₋₂₄ alkyl chain;
C, for each occurrence, independently comprises: a hydrophilic group comprising a hydroxide, an amine, a phosphate, a sulfur, and/or a sugar; a hydrophobic group comprising an amine, an amide, an ether, an ester, a N- or O-containing heterocycle, a thiol, a thioether, and/or a saturated or unsaturated C₁₋₂₄ alkyl chain; and/or one or more aromatic rings;
D, for each occurrence, independently is a branching unit comprising one or more alkyl chains, amides, ethers, esters, and amines, wherein the branching unit comprises 2 to 4 branches; and
m, for each occurrence, independently is 0 or 1.

35. The method of claim 34, wherein the oligonucleotide conjugate has a structure of Formula II: 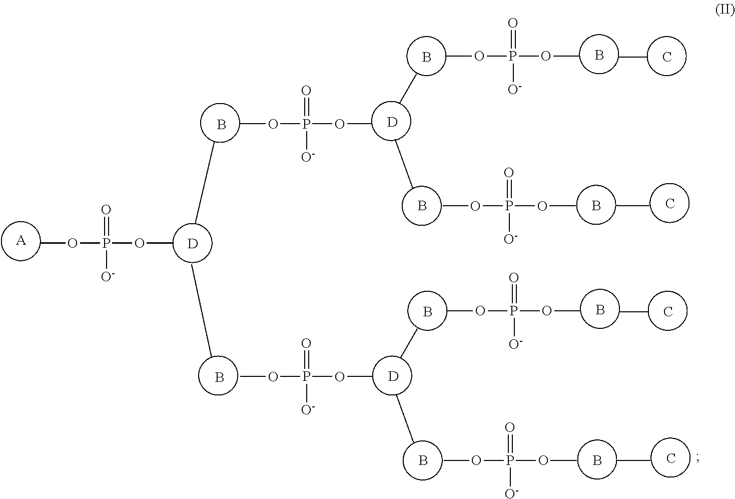
or
the oligonucleotide conjugate has a structure of Formula III: 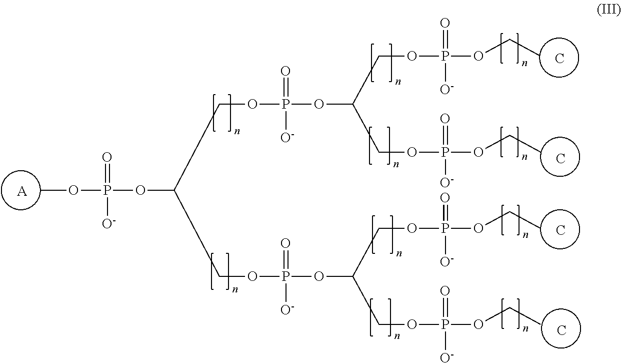
wherein n, for each occurrence, independently is an integer between 1-24; or
C is OH and n, for each occurrence, independently is 1, 6, or 12; or
the oligonucleotide conjugate has the structure of Formula IV: 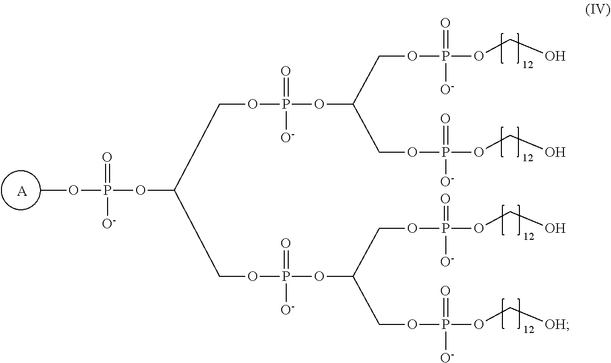
or
the oligonucleotide conjugate has a structure of Formula V:

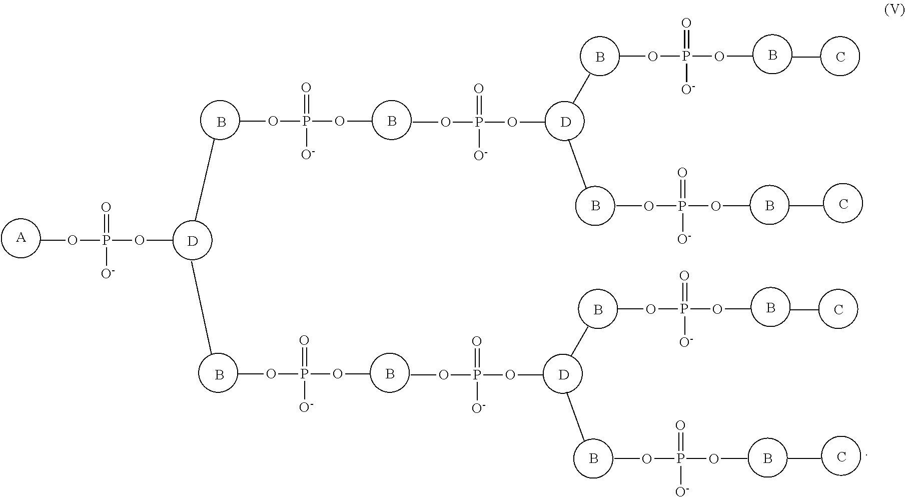

36-39. (canceled)

40. The method of claim 35, wherein the oligonucleotide conjugate has a structure of Formula VI: 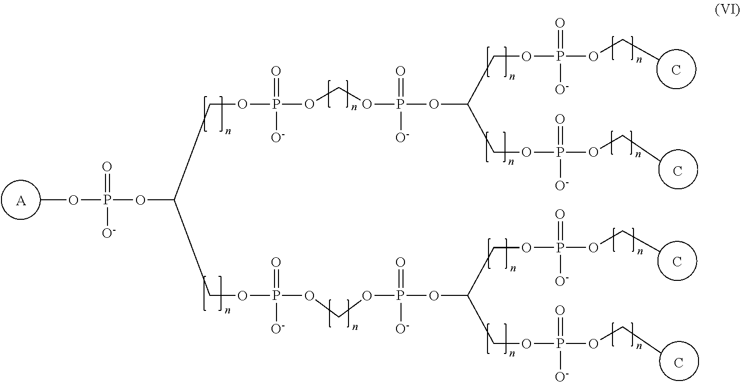
wherein n, for each occurrence, independently is an integer between 1-24, optionally wherein C is OH and n, for each occurrence, independently is 1, 6, or 12, optionally wherein the oligonucleotide conjugate has the structure of Formula VII:

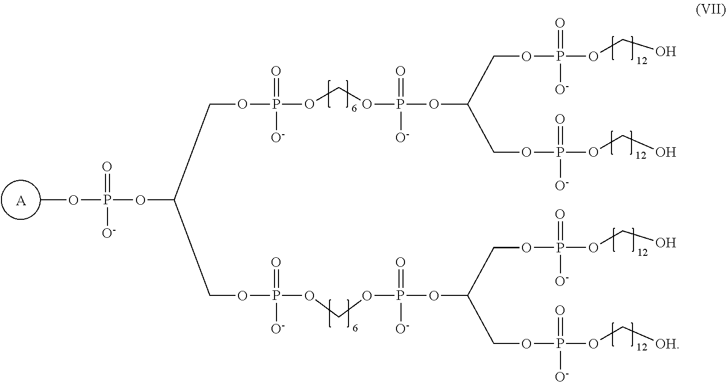

41-48. (canceled)

49. The method of claim 1, wherein the oligonucleotide has a sequence of any one of the sequences of SEQ ID NOs: 10 and 11.

50. (canceled)

51. A method for inhibiting the expression of an AIM2 gene in a cell, the method comprising:
(a) introducing into the cell an oligonucleotide conjugate comprising:
i) an oligonucleotide comprising a 5′ end, a 3′ end, and complementarity to a target nucleic acid; and
ii) a dendron that is linked to the oligonucleotide and comprises an end group, a phosphate group, and/or a hydrophobic chain; and
(b) maintaining the cell produced in step (a) for a time sufficient to obtain degradation of a mRNA transcript of the AIM2 gene, thereby inhibiting expression of the AIM2 gene in the cell.

52. An oligonucleotide conjugate for use in the treatment of cancer in a patient in need of such treatment, wherein the oligonucleotide conjugate is formulated for tumor delivery, and wherein the oligonucleotide conjugate comprises:
(a) an oligonucleotide comprising a 5′ end, a 3′ end, and complementarity to a target nucleic acid; and
(b) a dendron that is linked to the oligonucleotide and comprises an end group, a phosphate group, and/or a hydrophobic chain.

53. A pharmaceutical composition for treating cancer in a patient in need of such treatment, comprising:
an oligonucleotide conjugate comprising:
(a) an oligonucleotide comprising a 5′ end, a 3′ end, and complementarity to a target nucleic acid; and
(b) a dendron that is linked to the oligonucleotide and comprises an end group, a phosphate group, and/or a hydrophobic chain; and
a pharmaceutically acceptable carrier,
wherein the pharmaceutical composition is formulated for tumor delivery.

54. A combination comprising an oligonucleotide conjugate and an immune checkpoint inhibitor, wherein the oligonucleotide conjugate comprises:
(a) an oligonucleotide comprising a 5′ end, a 3′ end, and complementarity to a target nucleic acid; and
(b) a dendron that is linked to the oligonucleotide and comprises an end group, a phosphate group, and/or a hydrophobic chain.

55-56. (canceled)
